(19) United States
(12) Patent Application Publication
(10) Pub. No.: US 2025/0381364 A1
(43) Pub. Date: Dec. 18, 2025

(54) COMMUNICATION APPARATUS, METHODS, AND SYSTEMS OPERABLE TO ADMINISTER ENERGY PRESCRIPTIONS AND VERIFY THEIR EFFICACY

(71) Applicant: DataFeel Inc., Washington, DC (US)

(72) Inventor: Matthew Robert Leaper, Omaha, NE (US)

(73) Assignee: DataFeel Inc., Washington, DC (US)

(21) Appl. No.: 18/879,809

(22) PCT Filed: Jun. 29, 2023
(86) PCT No.: PCT/US23/26657

§ 371 Date: Dec. 30, 2024

Related U.S. Application Data
(60) Provisional application No. 63/356,950, filed on Jun. 29, 2022.

(51) Int. Cl. A61M 21/02; A61M 21/00; G06F 3/01

(52) U.S. Cl. CPC A61M 21/02; G06F 3/015; A61M 2021/0022; A61M 2021/0038; A61M 2021/0072; A61M 2205/3306; A61M 2205/332; A61M 2205/3368; A61M 2210/06; A61M 2210/083; A61M 2210/10; A61M 2230/04; A61M 2230/50; A61M 2230/63

(57) ABSTRACT

One aspect described herein is a data communication apparatus. The apparatus may comprise: electronic components operable to gather and output different types of physiological measurements associated with a user's chest, the electronic components comprising a multimodal sensor array, a transceiver, and a power source; and a housing that contains the electronic components and comprises an attachment element operable to maintain an alignment of the multimodal sensor array over the user's chest, wherein, when the housing is maintained over the user's chest, the multimodal sensor array is operable with the power source to measure the different types of physiological measurements, and the transceiver is operable with the power source and the sensor array to output the different types of physiological measurements to an external device. Related apparatus, devices, methods, and systems also are described.

### CROSS-REFERENCE TO RELATED APPLICATIONS

This application is a § 371 National Stage Entry of International Patent Application No. PCT/US23/26657, filed Jun. 29, 2023, claiming the benefit of priority of U.S. Provisional Patent Application No. 63/356,950, filed Jun. 29, 2022, the entireties of which are incorporated by reference into this application.

### TECHNICAL FIELD

Aspects of the present disclosure generally relate to communication apparatus, methods, and systems. Some aspects are described with reference to examples operable to administer energy prescriptions and verify their efficacy.

### BACKGROUND OF THE INVENTION

Some areas of a living body are commonly used for mounting objects, such as cars, fingernails, and nostrils. Many electronic devices are now small enough for mounting to the body in like fashion, making those locations equally desirable. Networks of electronic audiovisual communication devices may be utilized to create unique sensory experiences, such as the many instruments, screens, and speakers that are commonly necessary to host most any music event. There are many such examples of network benefits.

Certain lifestyle, medical, mental health, and wellness-directed benefits may be realized utilizing smaller, body-mounted audiovisual communication devices operable to create similarly immersive forms of unique sensory experiences on their own, without a screen; and through operation with an augmented or virtual reality system and/or any other type of visual display device. Further technological improvements are required to realize additional lifestyle, medical, mental health, and/or wellness-directed benefits.

### SUMMARY

Numerous aspects are described in this disclosure. One aspect described herein is a method for energy delivery to a user's body. For example, the method may comprise placing a first data communication device in contact with a first body part; placing a second data communication device in contact with a second body part; communicatively coupling the first data communication device to the second data communication device; generating a first energy signal at the first data communication device and delivering the first energy signal to the first body part; and generating a second energy signal at the second data communication device and delivering the second energy signal to the second body part, wherein the first energy signal is dependent on the second energy signal and operable therewith to simultaneously affect first nerves associated with the first body part and second nerves associated with the second body part.

By way of example, the method also may comprise: placing a first data communication device in contact with a first body part; placing a second data communication device in contact with a second body part; communicatively coupling the first data communication device and the second data communication device with a controller; generating, with one or both of the first data communication device and the second data communication device, physiological data associated with the user; receiving, with the controller, the physiological data; generating, with the controller or a processor in data communication therewith, a control signal based on the physiological data; causing, with the controller, the first data communication device to generate and output a first energy signal to the first body part responsive to the control signal; and causing, with the controller, the second data communication device to generate and output a second energy signal to the second body part responsive to the control signal, wherein combinations of the first energy signal and second energy signal are output during a treatment period as part of a synchronized communication to change in the physiological data by affecting nerves associated with the first body part and the second body part.

A strength of the first energy signal may be dependent on a strength of the second energy signal. A duration of the first energy signal may be dependent on a duration of the second energy signal. The step of placing the first data communication device may comprise placing the first communication device on the user's fingernail. The step of placing the second data communication device may comprise placing the second communication device on the user's head. The step of communicatively coupling the first data communication device to the second data communication device may comprise coupling the first data communication device to second data communication device via a wireless network.

The method may comprise placing a third data communication device in contact with a third body part; placing a fourth data communication device in contact with a fourth body part; communicatively coupling the first, second, third, and fourth data communication devices to one another; generating a third energy signal at the third data communication device and delivering the third energy signal to the third body part; and generating a fourth energy signal at the fourth data communication device and delivering the fourth energy signal to the fourth body part, wherein combinations of the first, second, third, and fourth energy signals are output during a treatment period as part of the synchronized communication operable to change in the physiological data by affecting nerves associated with the first, second, third, and fourth body parts.

The method may comprise placing a third data communication device in contact with a third body part; placing a fourth data communication device in contact with a fourth body part; communicatively coupling the first, second, third, and fourth data communication devices with the controller; causing, with the controller, the third data communication device to generate and output a third energy signal to the third body part responsive to the control signal; and causing, with the controller, the fourth data communication device to generate and output a fourth energy signal to the fourth body part responsive to the control signal, wherein the first, second, third, and fourth energy signals are output during the treatment period as part of the synchronized communication to change in the physiological data by affecting nerves associated with the first, second, third, and fourth body parts.

The step of placing the third data communication device may comprise placing the third communication device on the user's chest. The step of placing the fourth data communication device may comprise placing the second communication device on the user's arm. The first and second energy signals may comprise any of a heat energy signal, light energy signal, mechanical energy signal, haptic energy signal and acoustic energy signal. The acoustic energy signal may be an ultrasonic energy signal.

As a further example, the method also may comprise: placing a first data communication device in contact with a first body part, the first data communication device comprising a first housing, a first PCB that mechanically supports and electrically connects first electrical components in the first housing, the first electrical components comprising a first battery operable to power the first data communication device during a treatment period; placing a second data communication device in contact with a second body part comprising an artery, the second data communication device comprising a second housing, a second PCB that mechanically supports and electrically connects the second electrical components in the second housing, the second electrical components comprising a second battery operable to power the second data communication device during the treatment period; communicatively coupling the first data communication device and the second data communication device with a controller; generating, with one or both of the first data communication device and the second data communication device, physiological data associated with the user; receiving, with the controller, the physiological data; generating, with the controller or a processor in data communication therewith, a control signal based on the physiological data; causing, with the controller and the first battery, the first data communication device to generate and output a first energy signal to the first body part responsive to the control signal, the first energy signal comprising a frequency of vibration that affects a state of the user's brain via nerves associated with skin of the first body part; and causing, with the controller and the second battery, the second data communication device to generate and output a second energy signal to the second body part responsive to the control signal, the second energy signal comprising a heat flux that affects a temperature of blood flowing through the artery and a wavelength of light that affects an oxygenation level of the blood, wherein the first energy signal and second energy signal are output during a treatment period as part of a synchronized communication to change the physiological data by affecting the user's brain via the nerves associated with the first body part and the blood flowing through the artery of the second body part.

Another aspect described herein is a system for data communication. For example, the system may comprise a first data communication device in contact with a user's fingernail, the first data communication device including a first sensor operable to communicate first data associated with the user's fingernail to a master controller over a wireless network; a second data communication device in contact with the user's head, the second data communication device including a second sensor operable to communicate second data associated with the user's head to the master controller over the wireless network, and a third data communication device in contact with the user's chest, the third data communication device including a third sensor operable to communication second data associated with the user's chest to the master controller over the wireless network, wherein the first data communication device, the second data communication device and the third data communication device may be in communication with each other and operable to receive a unified control signal from the master controller via the wireless network.

By way of example, the system may comprise a first data communication device comprising a nail body positionable adjacent a nail plate the first sensor may be at least partially sealed in the nail body. The system may comprise a nail body comprised of a biocompatible material. The system may comprise a nail body comprised of one or more of a plurality of holes operable to house and connect the data communication device. The system may comprise a first data communication device comprising a first power source; a first controller operable to communicate over the wireless network associated with the nail plate; and a first energy generator operable to output an energy signal, wherein the first power source, the first controller, and the first energy generator are at least partially sealed in the nail body. The system may comprise a first power source comprising a lithium-ion battery that is rechargeable through or removably attachable from the nail body. The system may comprise a first controller comprising a first transceiver operable to communicate the first data to the master controller via the wireless network.

The system may comprise a first energy generator comprising a single-energy haptic generator operable to output one type of energy toward the nail bed. The system may comprise a first energy generator comprising an multi energy generator operable to output a plurality of different energies toward the nail bed. The system may comprise a first sensor of one of an Inertial Measurement Unit, a movement sensor, or a kinetic energy sensor. The system may comprise a first data communication device comprising a display element mounted on an outward-facing surface of the nail body. The system may comprise a display element comprising an LED screen. The system may comprise a display element cantilevering beyond a fingertip of the user's fingernail. The system may comprise an attachment element operable to secure the nail body to distal phalanx or a proximal phalanx. The system may comprise a plurality of the first data communication devices in contact a plurality of user's fingernails.

The system may comprise a second data communication device comprising a second housing that is adherable to the user's head the first sensor is contained in the housing. The system may comprise a housing comprising an attachment element operable to adhere the second housing to the user's head. The system may comprise an attachment element that is an adhesive tape. The system may comprise a second sensor comprising an optical sensor, a temperature sensor, or an ultrasonic transducer. The system may comprise a second sensor comprising an inertial measurement unit, a movement sensor, or a kinetic energy sensor. The system may comprise a second data communication device comprising a second energy generator. The system may comprise a second energy generator comprising a single-energy haptic generator operable to output one type of energy toward the user's head. The system may comprise a second energy generator comprising an multi energy generator operable to output a plurality of different energies toward the user's head. The system may comprise a second power source. The system may comprise a second power source comprising a lithium-ion battery that is removably attachable from the apparatus. The system may comprise a second energy generator comprising an LED operable to output light toward the user's head. 38.

The third data communication device may comprise a third housing in contact with the user's chest. The system may comprise a third housing comprising an attachment element to secure the housing to the user's chest. The system may comprise an attachment element that is an adhesive tape. The system may comprise a third sensor comprising a camera. The system may comprise a camera comprising a forward-facing camera and an upward facing camera. The system may comprise a third sensor comprising an inertial measurement unit, a movement sensor, or kinetic energy sensor. The system may comprise a third sensor comprising a microphone. The system may comprise a connecting element extending between the third housing and a support structure configured to be disposed on the user's neck. The system may comprise a support structure comprising a single-energy haptic generator operable to output one type of energy toward the user's neck. The system may comprise a support structure comprising a multi energy generator operable to output a plurality of different energies toward the user's neck. The system may comprise a support structure comprising plurality of multi energy generators. The system may comprise a connecting element as a lanyard. The system may comprise a third power source. The system may comprise a power source comprising a lithium-ion battery that is removably attachable from the apparatus.

The system may comprise a fourth data communication device comprising: a fourth housing; electronic components sealed in the housing, the electronic components comprising: a fourth sensor operable to communicate data associated with the user's environment through the housing to the master controller, a fourth energy generator operable to output a vibrational energy through the housing, and an LED operable to output light and heat energy through the housing.

The system may comprise a fourth sensor comprising an inertial measurement unit, a movement sensor, or a kinetic energy sensor. The system may comprise a fourth housing that is translucent. The system may comprise a fourth data communication device comprising a heat transfer clement operable to transfer heat from the LED to the fourth housing. The system may comprise a heat transfer clement that is an annular ring extending through the fourth housing to surround the LED. The system may comprise a heat transfer element connected to a fourth energy generator and operable transfer the vibrational energy to the fourth housing. The system may comprise a heat transfer clement that contacts a fourth energy generator. The system may comprise a heat transfer clement contacting an inner frame of the data communication device. The system may comprise an outer surface of the data communication device comprise a lens operable to diffuse the light. The system may comprise an LED that is a multi-color LED.

As a further example, the system also may comprise: a first data communication device in contact with a user's fingernail, the first data communication device comprising a first housing, a first PCB that mechanically supports and electrically connects first electrical components in the first housing, the first electrical components including a first sensor operable to communicate first data associated with the user's fingernail to a master controller over a wireless network, a first energy generator operable with the master controller to affect the user's brain by outputting a first energy that affects a state of the user's brain via nerves proximate to the user's fingernail, and a first battery operable to power the first data communication device during a treatment period; a second data communication device in contact with the user's head or neck, the second data communication device comprising a second housing, a second PCB that mechanically supports and electrically connects the second electrical components in the second housing, the second electrical components including a second sensor operable to communicate second data associated with the user's head to the master controller over the wireless network, a second energy generator operable with the master controller to affect blood flowing into the user's brain by outputting a heat flux to blood flowing through an artery of the user's head or neck and a wavelength of light that affects an oxygenation level of the blood; and a third data communication device in contact with the user's chest, the third data communication device including a third sensor operable to communication audio data associated with the user's chest to the master controller over the wireless network and a third battery operable to power the third data communication device during the treatment period, wherein the first data communication device, the second data communication device, and the third data communication device are in communication with each other and operable to receive a unified control signal from the master controller via the wireless network and activate the first energy and second energy generators responsive to unified control signal.

Another aspect described herein is an apparatus for data communication. For example, the apparatus may comprise an apparatus comprising a housing maintainable against a user's chest; and a sensor array that is mounted to housing and operable to: generate physiological data associated with the user's heart or lungs comprising acoustic data; and communicate the physiological data to one or more processors over a data communication network.

By way of example, the housing may comprise an attachment element to secure the housing to the user's chest. The attachment element may comprise an adhesive tape. The sensor array may comprise a camera. The camera may comprise a forward-facing camera operable to capture environmental data associated with the user and/or an upward facing camera operable to capture facial data associated with the user. The sensor array may comprise one of an inertial measurement unit, a movement sensor, and a kinetic energy sensor.

The sensor array may comprise a microphone. For example, the microphone may comprise: a first microphone positioned for generating first acoustic data associated with the user's right lung, a second microphone positioned for generating second acoustic data associated with the right side of the user's heart, a third microphone positioned for generating third acoustic data associated with the user's left lung, and a fourth microphone positioned for generating fourth audio data associated with the left side of the user's heart. The sensor array may comprise electrodes. For example, the electrodes may comprise a first electrode positioned for generating first electrical data associated with the user's right lung, a second electrode positioned for generating second electrical data associated with the right side of the user's heart, a third electrode positioned for generating third electrical data associated with the user's left lung, and a fourth electrode positioned for generating fourth electrical data associated with the left side of the user's heart.

The apparatus may comprise a connecting element that extending between the housing and a support structure. The support structure may be supportable from the user's neck to position the sensor unit over the user's chest at a location adjacent the user's heart and lungs. The support structure may comprise an energy generator in data communication with the one or more processors over a data communication network. The connecting element may comprise a lanyard. The multi-energy generator may be operable to output a plurality of different energies. For example, the plurality of different energies may comprise a vibrational energy and a thermal energy. The thermal energy may comprise hot or cold. The apparatus may comprise a power source and power source may comprise a lithium-ion battery removably attachable to the apparatus.

As a further example, the apparatus may comprise: electronic components operable to gather and output different types of physiological measurements associated with a user's chest, the electronic components comprising a multimodal sensor array, a transceiver, and a power source; and a housing that contains the electronic components and comprises an attachment element operable to maintain an alignment of the multimodal sensor array over the user's chest, wherein, when the housing is maintained over the user's chest, the sensor array is operable with the power source to measure the different types of physiological measurements, and the data transceiver is operable with the power source and the sensor array to output the different types of physiological measurements to an external device.

In this example, the multimodal sensor array may comprise left sensors located on a left side of the user's chest and right sensors located on a right side of the user's chest. The multimodal sensor array may comprise two or more of: a first sensor operable to measure a first type of physiological measurements associated with the user's heart; a second sensor operable to measure a second type of physiological measurements associated with the user's lungs; a third sensor operable to measure a third type of physiological measurements associated with the user's head; and a fourth sensor operable to measure a fourth type of physiological measurements associated with the user's body. The first sensor may comprise electrodes positioned to measure the first type of physiological measurements. The electrodes may comprise left electrodes located on a left side of the user's chest and right electrodes located on a right side of the user's chest; and the first type of physiological measurements may comprise a left measurement for the left side of the user's chest and a right measurement for the right side of the user's chest.

The second sensor may comprise a transducer positioned to measure the second type of physiological measurements. The transducer may comprise a left microphone located on a left side of the user's chest and a right microphone located on a right side of the user's chest; and the second type of physiological measurements may comprise a left measurement for the left side of the user's chest and a right measurement for the right side of the user's chest. The third sensor may comprise an input device positioned to measure the third type of physiological measurements. The input device may comprise a camera positioned to measure the third type of physiological measurements. The camera may comprise an upward-facing camera; and the third type of physiological measurements may comprise data associated with the user's face. The camera may comprise a forward-facing camera; and the third type of physiological measurements may comprise data associated with the user's environment. The fourth sensor may comprise an inertial measurement unit, a movement sensor, or a kinetic energy sensor; and the fourth type of physiological measurements may comprise data associated with a movement of the user's body.

The housing may comprise a radiotranslucent material and the transceiver may be operable to output the different types of physiological measurements to the external device through the radiotranslucent material. A portion of the housing may be translucent. The electronic components may be hermetically sealed in the housing to prevent exposure to water when removably attached to the skin. The attachment element may comprise a skin-facing surface of the housing with a biocompatible adhesive layer operable with the skin to maintain the alignment of the multimodal sensor array over the user's chest by acting on the skin. The skin-facing surface may be conformable against the user's chest to form an adhesive bond between the biocompatible adhesive layer and the skin. The apparatus may comprise flexible conductors that are embedded in the skin-facing surface and operable to establish a data connection between the multimodal sensor array and the transceiver. The multimodal sensor array may comprise left sensors located on a left side of the user's chest and right sensors located on a right side of the user's chest; the transceiver and the power source may be located between the left sensors and the rights sensors; and the flexible conductors may comprise left conductors connecting the left sensors to the transceiver and the power source and right conductors connecting the right sensors to the transceiver and the power source. The attachment element may comprise an elastic portion that is operable with the user's torso to maintain the alignment of the multimodal sensor array. The attachment element may comprise a lanyard that is operable with the user's neck to maintain the alignment of the multimodal sensor array. In this and related examples, when the housing is maintained over the user's chest, the multimodal sensor array, the transceiver, and the power source may be operable with the external device over a wireless network to determine a cardiovascular measure associated with the user's chest.

Another aspect described herein is another system for data communication. For example, the system may comprise a networked plurality of communication apparatus comprising one or more of: (I) a first communication apparatus comprising: a nail body adherable to a nail plate of a user; and a first data communication device mounted to the nail body comprising a first sensor operable to communicate first data associated with the user to a master controller; (II) a second communication apparatus comprising: a sensor housing adherable to the user's head; a second data communication device attached to the sensor housing and comprising a second sensor operable to communicate second data associated with the user to the master controller; and (III) a third communication apparatus comprising: a plate adherable to the user's chest; and a third data communication device attached to the plate and comprising a third sensor operable to communicate third data associated with the user to the master controller; and a fourth communication apparatus comprising: a light-transmitting housing; a fourth data communication device sealed in the light-transmitting housing and comprising: a sensor unit operable to communicate fourth data associated with the user through the housing to the master controller, a haptic energy generator operable to output a haptic energy through the housing, and a light generator operable to output light and heat energy through the housing, the networked plurality of communication apparatus being operable with the master controller to deliver an energy prescription to the user by activating a combination of the first apparatus, the second apparatus, the third apparatus, and the fourth apparatus in a coordinated manner during a treatment period as part of a synchronized energy treatment.

By way of example, the networked plurality of energy outputting technologies may be operable to deliver the energy prescription responsive to a unified control signal generated by the master controller. The fourth data communication device may be operable to output a chemical signal responsive to the unified control signal.

Related apparatus, methods, and systems also are described, each being operable individually and/or in combination with one another to administer energy prescriptions and verify their efficacy according to this disclosure.

### BRIEF DESCRIPTION OF THE DRAWINGS

The accompanying drawings are incorporated in and constitute a part of this specification. These drawings illustrate exemplary aspects of the present disclosure that, together with the written descriptions provided herein, serve to explain the principles of this disclosure. Numerous aspects are shown conceptually in the drawings and particularly described, pointed out, and taught in the written descriptions. Some structural and operational aspects may be better understood by referencing the written portions together with the accompanying drawings. For these reasons, a more complete appreciation of the subject matter of the present disclosure and the various advantages thereof may be realized by reference to the following detailed description, in which reference is made to the following accompanying drawings:

FIG. 1 is a diagram illustrating a communication system according to an embodiment of the present disclosure worn by a user;

FIG. 2 is a diagram illustrating a communication system according to another embodiment of the present disclosure worn by a user;

FIG. 3 is a diagram illustrating a communication system according to another embodiment of the present disclosure worn by a user;

FIG. 4 is a perspective view of a communication apparatus according to an embodiment of the present disclosure;

FIG. 5 is a cross-sectional view of the communication apparatus of FIG. 4 taken at Section A-A of FIG. 6;

FIG. 6 is a schematic diagram of the communication apparatus of FIG. 4 that shows Section A-A extending therethrough;

FIG. 7 is a cross-sectional view of a communication apparatus according to another embodiment of the present disclosure taken at Section B-B of FIG. 8;

FIG. 8 is a schematic diagram of the communication apparatus of FIG. 7 that shows Section B-B extending therethrough;

FIG. 9 is a perspective view of a communication apparatus according to another embodiment of the present disclosure;

FIG. 10 is a cross-sectional view of the communication apparatus of FIG. 9 taken at Section C-C of FIG. 11;

FIG. 11 is a schematic diagram of the communication apparatus of FIG. 9 that shows Section C-C extending therethrough;

FIG. 12 is a perspective view of a communication apparatus according to another embodiment of the present disclosure;

FIG. 13 is a cross-sectional view of the communication apparatus of FIG. 12 taken at Section D-D of FIG. 14;

FIG. 14 is a schematic diagram of the communication apparatus of FIG. 12 that shows Section D-D extending therethrough;

FIG. 15 is a perspective view of a communication apparatus according to another embodiment of the present disclosure;

FIG. 16 is a front view of a communication apparatus according to another embodiment of the present disclosure;

FIG. 17 is a side of the communication apparatus of FIG. 16;

FIG. 18 is side cross-sectional view of the communication apparatus of FIG. 16;

FIG. 19 is back view of the communication apparatus of FIG. 16;

FIG. 20 is a first schematic diagram of the communication apparatus of FIG. 16;

FIG. 21 is a second schematic diagram of the communication apparatus of FIG. 16;

FIG. 22 is a diagram illustrating a communication system according to another embodiment of the present disclosure worn by a user;

FIG. 23 is a front view of a communication apparatus according to another embodiment of the present disclosure;

FIG. 24 includes a side cross-sectional view (at left) and a partial side cross-section view (at right) of the communication apparatus of FIG. 23;

FIG. 25 is a first schematic diagram of the communication apparatus of FIG. 23;

FIG. 26 is a second schematic diagram of the communication apparatus of FIG. 23;

FIG. 27 is a front view of a communication apparatus according to another embodiment of the present disclosure;

FIG. 28 is a back view of the communication apparatus of FIG. 27;

FIG. 29 is a front view of a communication apparatus according to another embodiment of the present disclosure;

FIG. 30 is a perspective view of a communication apparatus according to another embodiment of the present disclosure;

FIG. 31 is a partial perspective view of the communication apparatus of FIG. 30;

FIG. 32 is a partial front view of the communication apparatus of FIG. 30;

FIG. 33 is a partial side cross-sectional view of the communication apparatus of FIG. 30;

FIG. 34 is a first schematic diagram of the communication apparatus of FIG. 30, and

FIG. 35 is a second schematic diagram of the communication apparatus of FIG. 30.

### DETAILED DESCRIPTION

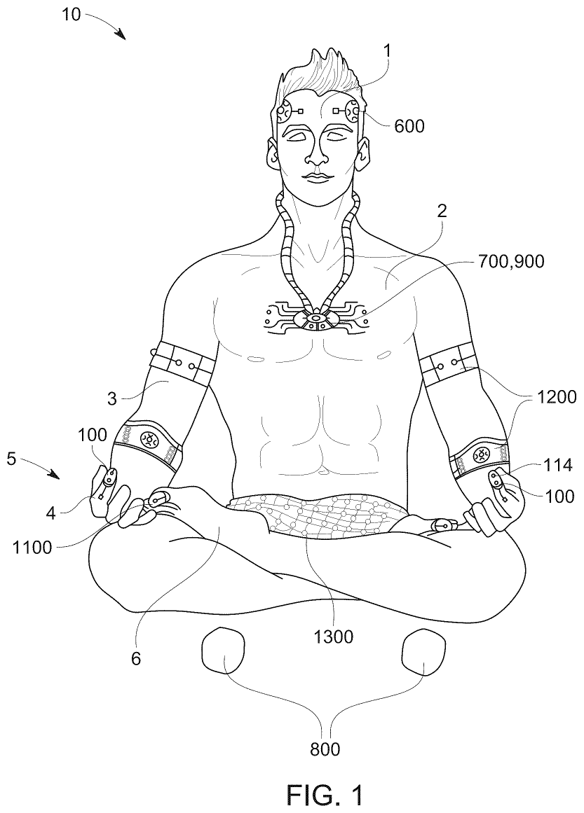

Aspects of the present disclosure are now described with reference to exemplary communication apparatus, methods, and systems, including examples operable to administer energy prescriptions. Some aspects may comprise an exemplary network of body mountable and/or wearable data communication devices, methods, and systems forming a multi-node array of audio, haptic, chemical (e.g., aroma sensation), and/or visual communication devices, in which each element may be networked together with each other and the internet. For example, an exemplary communication system 10 is shown in FIG. 1 to provide a conceptual overview of the various devices, methods, and system-level configurations described in detail in this disclosure. Any references to these exemplary concepts are provided for convenience and not intended to limit the present disclosure unless claimed. Accordingly, the aspects disclosed herein may be utilized for any analogous communication device, method, or system, including any type of body mountable, implantable, wearable, and networkable technology.

The terms “proximal” and “distal,” and their respective initials “P” and “D,” may be used to describe relative components and features. Proximal may refer to a position closer to, whereas distal may refer to a position further away. Appending the initials P or D to a number may signify its proximal or distal location or direction. Unless claimed, these directional terms are provided for convenience and not intended to limit this disclosure.

Aspects of this disclosure may be described with reference to one or more axes. An clement may extend along an axis, be moved along said axis in first or second direction, and/or be rotated about said axis in a first or second direction. For example, as described in some examples below, a fingernail element may extend along a digit axis, be moved along the digit axis when mounted onto a fingernail, and/or be movably fixed relative to the digit axis after being mounted onto the fingernail. One axis may intersect another axis, resulting in a transverse and/or perpendicular relationship therebetween. For example, two or three perpendicular axes may intersect at an origin point to define a Cartesian coordinate system. The directional terms proximal and distal may be used with reference to any axis. One axis may be a longitudinal axis extending along a length of an element, such as a central longitudinal axis extending along the length and through a centroid of the element.

Terms such as “may” and “can,” and like variation, are intended to describe optional aspects of the present disclosure, any of which may be covered by the claims set forth below. Terms such as “comprises,” “comprising,” or like variation, are intended to describe a non-exclusive inclusion, such that a device, method, or system comprising a list of elements does not include only those elements but may include other elements not expressly listed or inherent thereto. The term “and/or” indicates a potential combination, such that a first and/or second element may likewise be described as a first element, a second element, or a combination of the first and second elements. These potential combinations are provided as examples. Numerous other combinations are inherent to this disclosure. Unless stated otherwise, the term “exemplary” is used in the sense of “example” rather than “ideal.”

Aspects of this disclosure are directed to communication apparatus, devices, methods, and systems for delivering an “energy prescription” comprising one or more energy-based therapies deliverable to one or more locations of a user's body in a precise, repeatable way that allows for consistent therapeutic experiences between therapy sessions and across users. One example is a communication system comprising a networked group of energy outputting technologies that are wearable against the user's skin and operable to deliver the energy prescription by causing a plurality of communication apparatus located on or adjacent different portions of the skin to simultaneously output one or more different types of energy toward the user in a coordinated matter during a treatment period responsive to a unified control signal. For example, the energy prescription may be administered to the user as part of a synchronized communication of the different types of energy to different sets of nerves and tissues for the purpose of affecting the user's brain and/or mind by inducing different mental states, overwhelming the senses, or otherwise changing the user's brain activity. As a further example, the energy prescription may be administered for the purpose of affecting physiological data associated with the user, such as heart rate, blood oxygenation, blow flow, and the like.

In these examples, and others described herein, the unified control signal may comprise any data function or steam operable with the networked group of energy outputting technologies to administer the energy prescriptions by causing the synchronized outputs. Different format types may be used. For example, the unified control signal may comprise a MIDI or MPEG signal that is generated by an external device (e.g., a cloud computing platform) and distributed to one or more of the energy outputting technologies.

Different combinations of technologies may be utilized to output the different types of energy within this exemplary communication system, including technologies optimized to affect or communicate with (i) nerves associated with the eyes, cars, or nose of the user; (ii) nerves associated with the skin at different locations on the user's body, such as the digits, the temple, or the neck; and/or (iii) underlying tissues at the different locations, such as the mechanoreceptors at the finger tips, the temporal arteries at the temples, and muscles of the neck, tendons, bone, etc. Different single- and multi-energy technologies are described as being operable to output one and/or more different types of energy toward the user, including electrical energies, magnetic energies, optical energies, pressure energies, thermal energies, vibratory energies, and the like. Energy prescriptions may be administered to the user with synchronized outputs from any combination of these technologies, including any combination of: (i) single- or multi-energy haptic technologies operable responsive to the unified control signal to output different types of haptic energy to the skin; (ii) single- or multi-color LEDs operable responsive to the unified control signal to output different types of light energy to the eyes and/or skin; and/or (iii) single- or multi-chemical diffusers operable responsive to the unified control signal to output different types of chemicals into the user's environment, such as those affecting the user's sense of smell.

For each communication system described herein, the plurality of energy outputting technologies may comprise a plurality of different wearable and environmental apparatus located on or adjacent the user. The unified control signal may be sent wirelessly at regular intervals during each treatment period to cause particular outputs at particular locations and particular times as part of the synchronized treatment stimulus. For example, the synchronized treatment stimulus may comprise different patterns of outputs, including any combination of audio, haptic, aroma sensory, and/or visual energy outputs, each of which may be simultaneously output individually and/or in combination toward the user in a precise, repeatable way during the treatment period responsive to the unified control signal. As a further example, the patterns may comprise any type of frequency, pulsing, and/or other static or variable output of audio, haptic, aroma sensory, and/or visual energy from one or more different locations that also may be static or variable when relevant to the production of certain sensory experiences.

Aspects of the unified control signal may be driven by real-time data streaming services, such as those provided every day to millions of music subscribers worldwide; and/or most any other data streams, including those from augmented/virtual reality systems. Different portions of unified control may cause different communication apparatus to output different energies to different parts of the body at different frequencies, causing the brain to process the outputs as a difference of the frequencies, like a binaural beat, but with a more wholistic approach that goes beyond audio outputs. For example, the unified control signal may cause a first set of communication apparatus on the user's left side to output vibrational energies toward the user's skin at a first frequency of 300 Hertz and a second set of communication apparatus on the user's right side to output vibrational energies toward the user's skin at a second frequency of a second frequency of 250 Hertz so that user's brain gradually falls into synchrony with the difference, causing the user to perceive a synchronous frequency of a 50 Hz that is commonly associated with high levels of focus, making it possible for an attending therapist to utilize the communication system described herein administer this type of energy prescription to patients struggling with focus, such as those with ADHD.

Aspects of the unified control signal also may be driven by data associated with the user. For example, the communication systems described herein may comprise sensors operable to gather physiological data, environmental data, and/or other sensory data associated the user and modify the unified control signal responsive to the data. Different sensors may be mounted on different parts of the user's body and operable to gather different data associated with the user while the synchronized treatment stimulus is being delivered. All or portions of the physiological data may be utilized to verify an efficacy of the energy prescription. For example, the communication systems described herein may comprise computing technologies operable to analyze the physiological data, environmental data, and/or other sensory data to confirm that the energy prescription produced a desired or intended result during the treatment period. For example, while administering an energy prescription intended to have a calming effect on the user, these computing technologies may be operable to verify that the prescription is producing the desired effect by confirming in real-time that the user is calm because they are experiencing a decelerated heart and/or breathing rate responsive to the different energy outputs.

Different energy prescriptions may be administered according to this disclosure as physics-based alternatives to existing chemical and/or pharmaceutical treatments. For example, the communication apparatus, devices, methods, and systems described herein may be operable to administer different energy prescriptions for: (i) stochastic resonance treatments, wherein a quality of noise may be used to amplify a signal in the form of vibration and light to positively affect the somatosensory system, facilitating the stabilization of multiple biorhythms; (ii) neuropathy treatments, wherein combinations of transcutaneous electrical nerve stimulation (TENS), hot and cold therapy, and massage, are recognized as effective; and/or (iii) thermal treatments, in which thermal regulation is used to affect the affect the brain and nervous system in tandem with electrical and vibration therapies.

Exemplary aspects of different communication systems are now described with reference to a plurality of communication apparatus operable to administer energy prescriptions by outputting a synchronized treatment stimulus responsive to a unified control signal during a treatment period, causing one or more sensors to collect physiological data associated with the user during the treatment period, confirming that the synchronized treatment stimulus is having or had an intended effect on the user; and/or iterating the control signal based on the data.

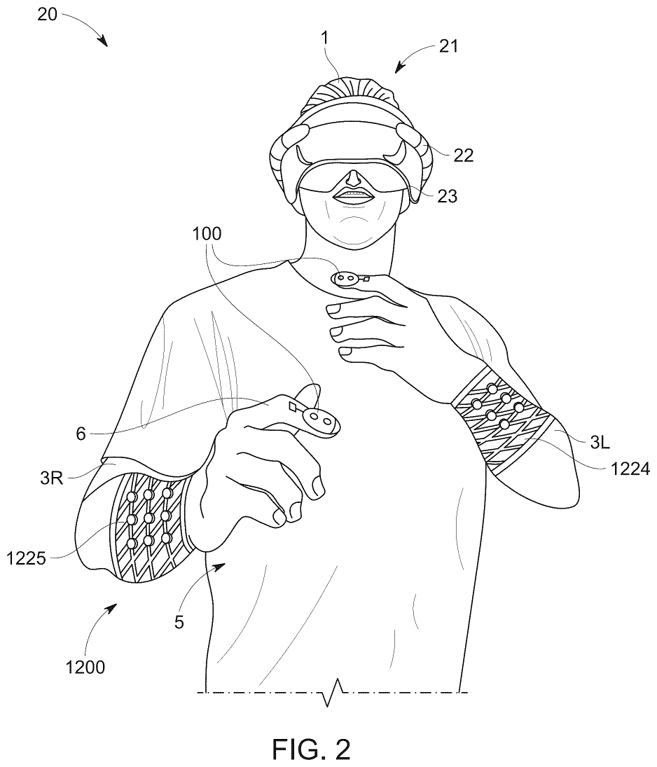

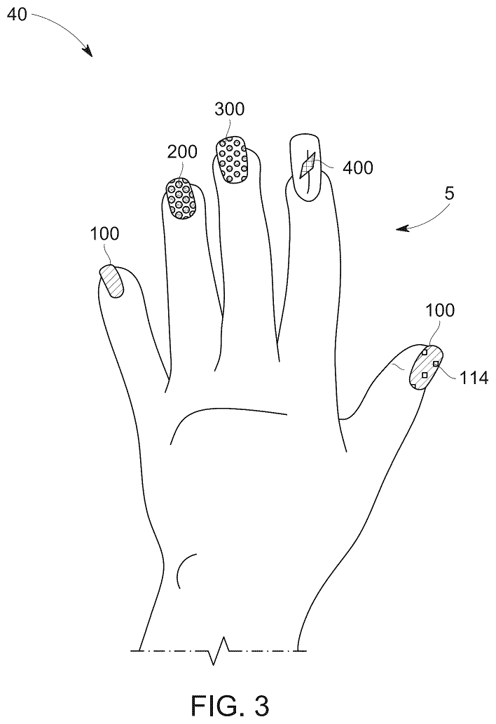

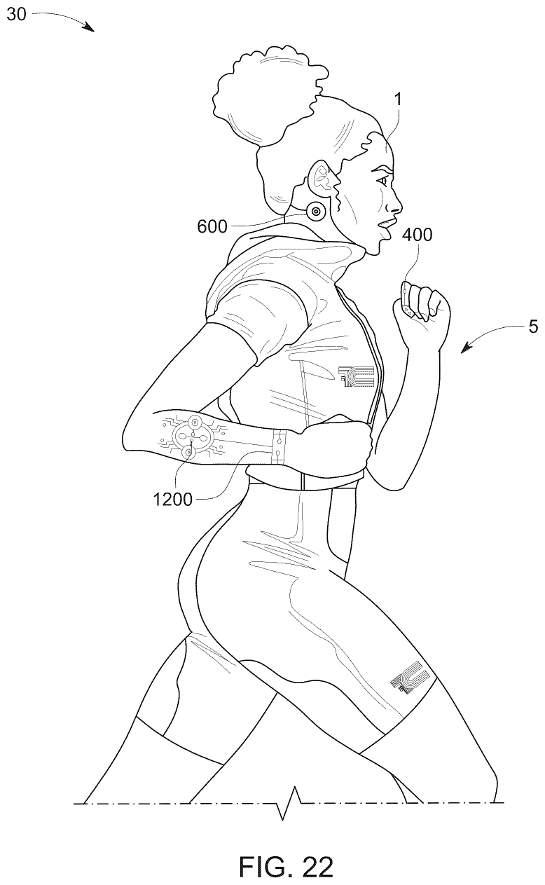

As shown in FIG. 1, for example, an exemplary communication system 10 may comprise a plurality of communication apparatus including one or more of: a digit-mounted apparatus 100, 200, 300, 400, 500, 1100; a head-mounted apparatus 600; a chest-mounted apparatus 700, 1000; a neck-mounted apparatus 900; a limb-mounted apparatus 1200; a fabric-integrated apparatus 1300; and/or a multi-purpose apparatus 800. Some aspects of each treatment apparatus 100, 200, 300, 400, 500, 600, 700, 800, 900, 1000, 1100, 1200, and/or 1300 are described with ongoing reference to communication system 10 and/or additional communication systems 20 (e.g., FIG. 2), 40 (e.g., FIG. 3) and/or 30 (e.g., FIG. 22) described herein, but could alternatively be described as stand-alone devices operable to output their respective energies responsive to an independent control signal(s).

Several operating and/or manufacturing methods are described with reference to communication systems 10, 20, 30, 40, and/or elements thereof. Aspects of any method steps, enabling structures, and/or functions described with reference to communication systems 10, 20, 30, and/or 40 or any of treatment apparatus 100, 200, 300, 400, 500, 600, 700, 800, 900, 1000, 1100, 1200, and/or 1300 are representative and not limiting unless claimed.

### Digit-Mounted Communication Apparatus, Methods, and Systems

Exemplary aspects of this disclosure are now described with reference to communication apparatus 100. As shown in FIGS. 3, 4, 5, and/or 6, communication apparatus 100 may comprise a nail body 110 and a data communication device 120 housed in nail body 110.

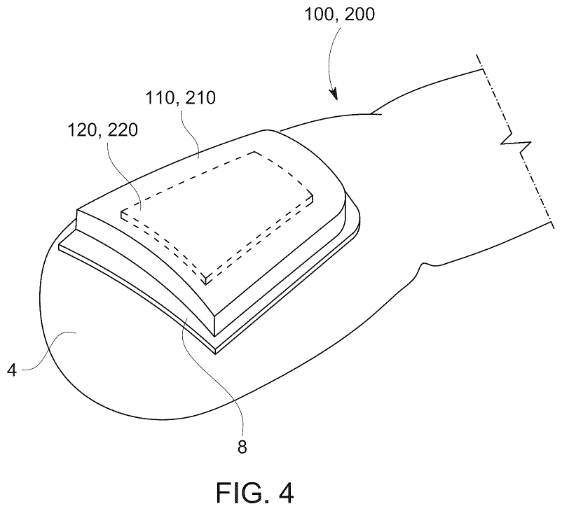

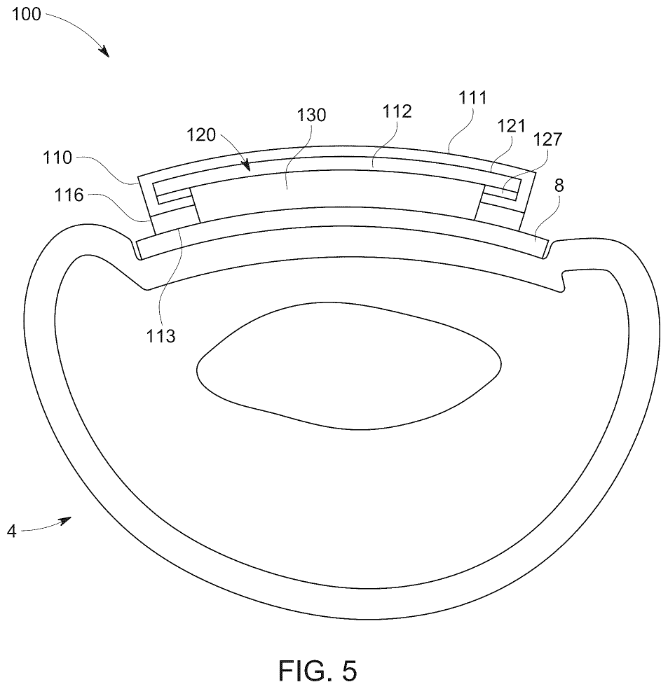

As shown in FIGS. 3, 4 and/or 5, nail body 110 may formed from a biocompatible material to have an elongated shape with a curvature shaped to conform against a natural curvature of a fingernail 8 of a human finger digit 4, making it body-mountable and wearable on a nail plate of fingernail 8. Nail body 110 may be formed form a rigid material. As shown in FIG. 5, nail body 110 may be formed around data communication device 120 with a molding method comprising placing data communication device 120 into a mold and flowing a molten amount of the biocompatible material (e.g., an acrylic thermoplastic) around communication device 120 in the mold, allowing nail body 110 to form around communication device 120 when the biocompatible material cools.

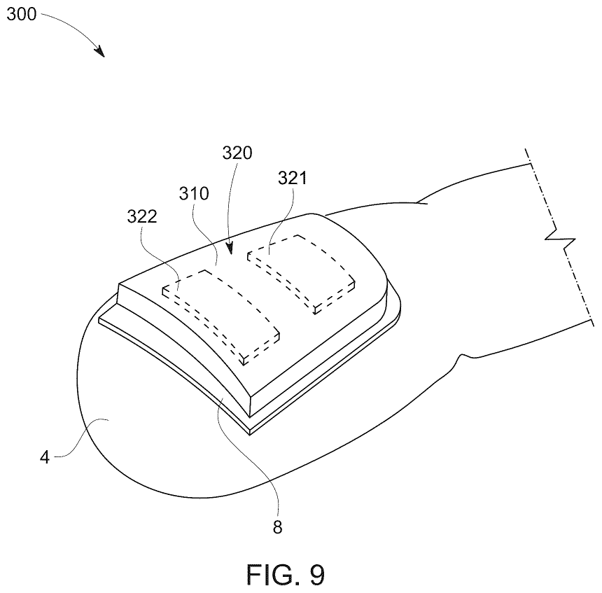

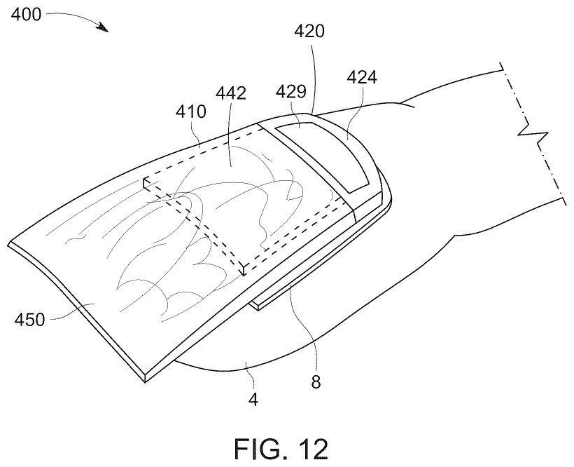

Aspects of nail body 110 and/or its materials may be optimized for enhanced structural strength relative to traditional acrylic nails. For example, if nail body 110 is shaped like that of an artificial nail historically worn on the finger digits, then a cross-sectional thickness and/or shape of nail body 110 may be sized and structurally able to rigidly cantilever outwardly from a distal end of fingernail 8 (e.g., like FIG. 12). Alternatively, as a further example, if nail body 110 is sized to fit within a length of fingernail 8 and thus shorter than a traditional artificial nail (e.g., like FIG. 9), then a cross-sectional thickness and/or shape of nail body 110 may conform with a shape of fingernail 8. The durability of nail body 110 may be optimized to enable and promote repeated uses of communication apparatus 100 with different users over an extend period of time. For example, communication apparatus 100 may be owned by a salon, leased to a first user when nail body 110 is adhered to the first user's finger and data communication device 120 is paired with their phone, recovered by the salon after a time (e.g., 3-7 days), cleaned with existing organic solvents (e.g., fingernail polish remover), recharged, and then leased to a second user when nail body 110 is adhered to the second user's finger and data communication device 110 is paired with their phone.

As shown in FIG. 5, nail body 110 may be formed from the biocompatible material to have a top surface 111, an interior cavity 112, and a bottom surface 113. Aspects of top surface 111 may be uniquely recognizable by a camera system that is powered by computer vision and operable to recognize certain types of shapes and patterns in a field of vision, much like those with augmented/virtual reality systems (AR/VR). As shown in FIG. 5, top surface 111 may comprise any type of adornments, exterior coatings, features, paints, surfaces, and/or structures operable to create and maintain any type of visual pattern that is machine-readable by cameras of the computer vision system. For example, the top surface 111 may comprise a QR code that is painted or printed on top surface 111 to enhance its readability with computer vision systems; light-reflecting materials positioned to reflecting red light back to the cameras; and the like.

An exemplary computer vision system is shown in FIG. 2 and described below with reference to an exemplary AR/VR system 21 comprising downward-facing cameras 23 positioned to view top surface 111 when communication apparatus 100 is worn on fingernail 8. In keeping with these examples, top surface 111 may comprise any machine-readable features, including any static or dynamic combination of colors, patterns, and/or physical shapes. Any machine-readable technologies may be similarly deployed. For example, the machine-readable features of top surface 111 also may comprise surface adornments 114, such as decorative elements like gems (e.g., FIG. 1), reflective and/or metallic surfaces (e.g., FIG. 3); visual output devices such as multi-color LEDs (e.g., FIG. 2) or LCDs (e.g., FIG. 12); and/or like features recognizable by cameras 23.

Interior cavity 112 may comprise interior portions of nail body 110 that are sized to house data communication device 120. In keeping with above, interior cavity 112 may be formed around data communication device 120 with nail body 110.

As show in FIG. 5, bottom surface 113 of nail body 110 may comprise an attachment element 116 operable to maintain a position of nail body 110 relative to fingernail 8. Attachment element 116 may comprise any known biocompatible adhesives, including those used with traditional artificial nails, such as professional nail glue like that sold by INFILILA®. The biocompatible adhesive may comprise a nail glue that is curable to attach bottom surface 113 to the top of fingernail 8 in semi-permanent manner allow for extended periods of operation without removal. Like a traditional adornment, nail body 110 may alternatively be attached to the nail plate of fingernail 8 by a layer of acrylic, gel, or powder, such as by pushing bottom surface 113 into the layer of acrylic, gel, or powder shortly after it has been applied to the nail plate and before it dries, cures, or otherwise solidifies into an adhesive element operable to attach nail body 110 to the nail plate. In addition, or alternatively still, attachment element 116 may comprise biocompatible adhesive layer (e.g., like a double-sided tape) so that bottom surface 113 may be removably attachable to fingernail 8, like Lee™ Press-On Nails™.

As shown in FIGS. 4, 5, and/or 6, data communication device 120 may be operable to generate sensory data associated with finger 4 and output the sensor data to an external device 11 over a wireless network connection 9. Because of its position on the nail bed of fingernail 8 of finger 4, data communication device 120 may be uniquely configured to gather movement, position, and/or physiological data associated with the tip of finger 4.

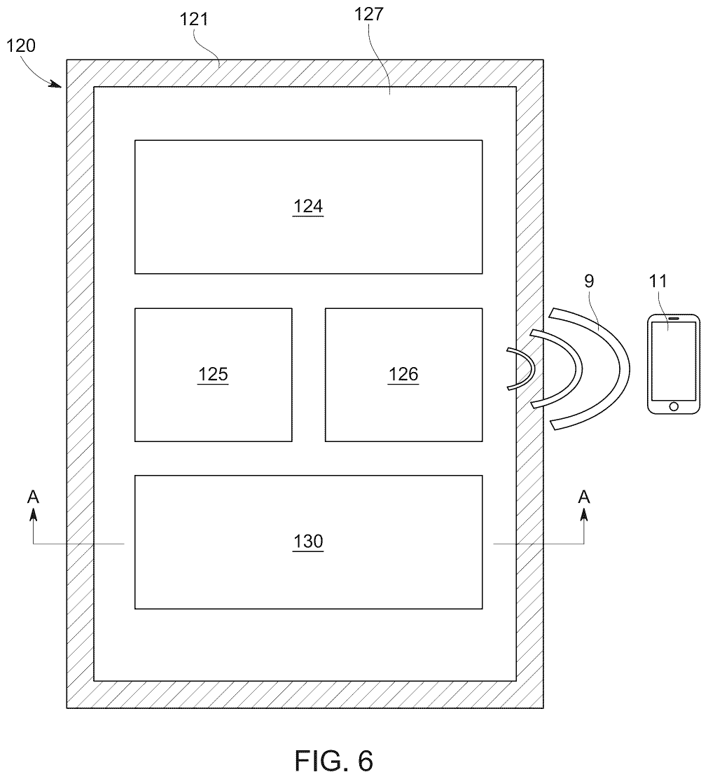

As shown in FIG. 6, data communication device 120 may comprise a housing 121, a power source 124, a controller 125, a data transceiver 126, a printed circuit board or “PCB” 127, and a sensor 130. As shown in FIGS. 5 and 6, housing 121 may comprise a thin layer of polymeric material that is formed to contain PCB 127 and each of power source 124, controller 125, data transceiver 126, and sensor 130 mounted thereto. If nail body 110 is formed with the molding method described above, the polymeric material of housing 121 may prevent these elements from being damaged by the molten biocompatible material during molding. Together, nail body 110 and housing 121 may hermetically seal data communication device 120 inside of interior cavity 112 so that communication apparatus 100 is durable enough for everyday use. Sidewalls of nail body 110 and housing 121 may allow data and power to pass therethrough. For example, both of nail body 110 and housing 121 may comprise “energy-translucent” material, such as a polymeric material that allows for wireless flows of data to/from data transceiver 126 (e.g., via Bluetooth or Wi-Fi) and electricity to power source 124 (e.g., via inductive charging). In this configuration, the electrical components of data communication device 120, i.c., power source 124, controller 125, data transceiver 126, PCB 127, and sensor 130 may thus be continuously operable and chargeable through nail body 110 when attached to fingernail 8.

Power source 124 may comprise one or more battery elements, such as a lithium-ion battery shaped to conform generally with a curvature of nail body 110. For example, power source 124 may comprise an elongated casing with a curved cross-sectional shape that is generally curved in one or more directions relative to the digit axis of finger 4, including one or more directions that are parallel or transverse relative to the digit axis of finger 4. Top surface 111 and/or bottom surface 113 of nail body 110 may be shaped to accommodate fingernail 8, whereas power source 124 may have a more general shape conducive to mass production and/or removability, as may any other element of data communication device 120.

Controller 125 may comprise any type of controller or microcontroller or other type of data processor and/or processing unit. As shown in FIG. 6, controller 125 may comprise any type of data communication, computation, and/or power transmission technologies operable to power sensor 130 with power source 124, receive sensory data from sensor 130, direct the sensory data to data transceiver 126, and/or cause data transceiver 126 to output the sensory data to external device 11 over wireless network 9.

Data transceiver 126 may comprise any wireless data communication technology operable with power source 124 and controller 125 to establish communications with wireless network 9 and/or external device 11 (e.g., via pairing) and/or output the sensory data to external device 11 over wireless network 9. The wireless data communication technology may comprise communication method enabled by Bluetooth®, Wi-Fi, and/or any other means for sending and receiving data between data processors, such as over the internet. For example, data transceiver 126 also may comprise a cellular chip like the like of Snapdragon® chips sold by Qualcomm®.

As shown in FIG. 6, PCB 127 may be operable to mechanically support and electrically connect power source 124, controller 125, data transceiver 126, and sensor 130 to housing 121 and nail body 110. PCB 127 may comprise a structural frame including conductive tracks, pads and other features etched from one or more sheet layers of copper laminated onto and/or between sheet layers of a non-conductive substrate. As shown in FIGS. 5 and/or 6, components 124, 125, 126, and 130 of data communication device 120 may be soldered onto the frame. As shown in FIGS. 4 and 5, perimeter edges of this frame may be engageable with interior surfaces of housing 121 so that device 120 may be fully contained therein.

Sensor 130 may be operable to generate, store, and/or output a stream data associated with a physical and/or physiological aspect of the user. For example, sensor 130 may comprise one or more electronic sensing devices that are mounted to PCB 127 and operable with power source 124, controller 125, and/or operable with data transceiver to generate sensory data associated with finger 4 and output that data to external device 11. In this example, sensor 130 may thus be incorporated into nail body 110, configured as above to be wearable on any finger 4 of hand 5, and operable from within interior cavity 112 to output one or more types of sensory data. As shown in FIG. 6, sensor 130 may comprise a movement sensor such as an inertial measurement unit, such as an electronic device that is operable with controller 125 and data transceiver 126 to measure and report a specific force of nail body 110, an angular rate of nail body 110, and/or an orientation of nail body 110, using any combination of accelerometers, gyroscopes, and/or magnetometers. In keeping with these examples, sensor 130 may be operable to generate data associated with a physical aspect of the user, such as the movement of finger 4 relative to space and/or another finger. In this way, sensor 130 and/or controller 125 may be configured to collect data associated with user 1 and output the collected to data transceiver 126 for output over wireless network 9 to external device 11. As described herein, sensor 130 may comprise an inertial measurement unit, like those sold within MICROSTRAIN® line sold by the Parker Hannifin corporation under the name 3DM®CX5-IMU available at https://www.microstrain.com/inertial-sensors/imu.

Additional aspects of communication apparatus 100 are now described with reference to a communication apparatus 200, a communication apparatus 300, a communication apparatus 400, a communication apparatus 500, and a communication apparatus 1100. Any aspects described with reference to one of apparatus 100, 200, 300, 400, 500, and/or 1100 may be similarly described with reference to another one of apparatus 100, 200, 300, 400, 500, and/or 1100, and vice versa, whether or not those aspects are referenced directly below. Aspects of apparatus 100, 200, 300, 400, 500, and/or 1110 may thus be interchangeably combined in keeping with this disclosure, each possible combination and/or iteration being part of this disclosure.

Exemplary aspects of this disclosure are now described with reference to communication apparatus 200. As shown in FIGS. 3, 4, 7, and/or 8, communication apparatus 200 may comprise a nail body 210 and a data communication device 220 housed in the nail body 210.

As shown in FIGS. 3, 4, and/or 7, nail body 210 may be similar or identical to nail body 110. For example, nail body 210 may comprise an elongated shape, curvature, and durability similar to that of nail body 110, making it similarly mountable and wearable. As shown in FIGS. 4 and/or 7, nail body 210 may comprise a top surface 211, an interior cavity 212, and a bottom surface 213 that are similar or identical to top surface 111, interior cavity 112, and bottom surface 113; and interior cavity 212 may be similarly operable to house and facilitate interconnections between electronic components of data communication device 220, permit operation of its electronic components, and enable repeated uses of communication apparatus 200 with different users over an extend period of time.

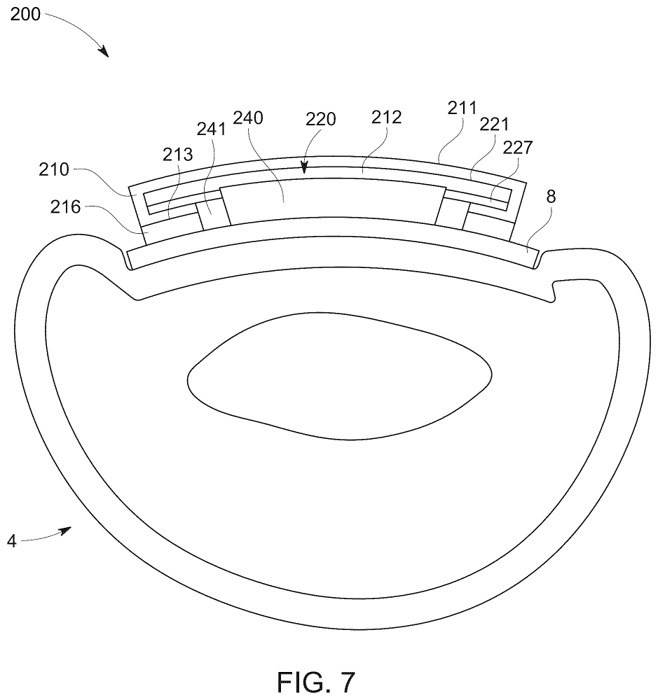

As shown in FIGS. 4, 7, and/or 8, data communication device 220 may be operable to input control data from external device 11 over wireless network connection 9 and output one or more different energies to finger 4 responsive to the control data. Because of its position on the nail bed of fingernail 8 of finger 4, data communication device 220 may be uniquely configured to communicate with user 1's brain via mechanoreceptors and like nerves associated with the tip of finger 4 at locations palmar of the nail bed.

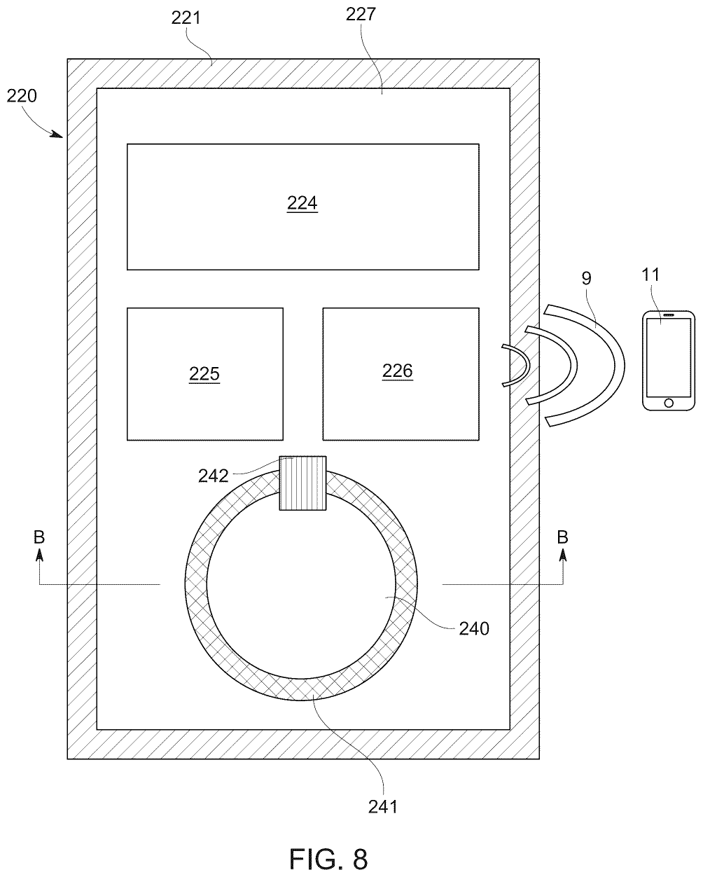

As shown in FIG. 8, data communication device 220 may comprise a housing 221, a power source 224, a controller 225, a data transceiver 226, a PCB 227, a bus 242, a haptic generator 240, and an isolating structure 241. Housing 221 may be similar or identical to housing 121 described above. For example, housing 221 may similarly comprise an energy-translucent material (e.g., a polymeric material) operable to define interior cavity 212; house PCB 227 and each of power source 224, controller 225, data transceiver 226, and haptic generator 240 mounted thereto; and be operable with nail body 210 to hermetically seal those elements within interior cavity 212.

Power source 224 may similarly comprise a lithium-ion battery and an inductive charging coil operable to charge the battery through nail body 210 and housing 221. As shown in FIG. 7, housing 221 may comprise exterior surfaces and sidewalls that hermetically seal power source 224, controller 225, data transceiver 226, and PCB 227 within interior cavity 212, thereby preventing them from exposure to fouling agents like dirt and liquids while simultaneously allowing data and power to pass therethrough.

Controller 225 and data transceiver 226 may be similar to controller 125 and data transceiver 126 described above, but with additional components for operating haptic generator 240. For example, data transceiver 226 may be operable to communicate and receive control data over wireless network 9 (e.g., from external device 11) and controller 225 may comprise additional circuitry for directing power from power source 224 to haptic generator 240 responsive to the control data. Haptic generator 240 may be operable with controller 225 to output one or more different energy types toward the nail plate of fingernail 8. For example, haptic generator 240 may comprise a single-energy haptic generator like those within the Precision Haptic™ line sold by Precision Industries™, a piezoelectric actuator like those within the PiezoHapt™ or PowerHap™ lines sold by the TDK Corporation; or a multi-energy haptic generator like those described in U.S. Pat. No. 10,959,674 issued Mar. 30, 2021, and International Patent Application No. PCT/US21/30528, filed May 3, 2021, the entireties of which are hereby incorporated by reference into this application.

As shown in FIG. 8, isolation structure 241 may be operable with haptic generator 240 to direct flows of the one or more energies toward fingernail 8 and limit flows of the one or more energies in directions toward other components mounted on PCB 227, such as toward power source 224, controller 225, and data transceiver 226, each of which may be affected by the energies at certain output levels of haptic generator 240. Isolation structure 241 may thus be described as an “energy break” that is operable with PCB 227 to maximize flows of energies output toward fingernail 8 from haptic generator 240 while simultaneously preventing those energies from negatively affecting other components mounted to PCB 227, such as power source 224, controller 225, and data transceiver 226. A material composition and/or width of isolation structure 241 may be selected to maximize the efficiency haptic generator 240. As shown in FIGS. 7 and 8, isolation structure 241 may comprise a 1.00 mm to 3.0 mm thick layer of natural rubber, neoprene, and/or butyl rubber located between interior surfaces of hole extending through PCB 227 and exterior surfaces haptic generator 240, such as a ring-like seal strip that wraps around haptic generator 240 and is mountable therewith.

As shown in FIG. 7, a nail-contacting portion of haptic energy generator 240 may extend from interior cavity 212 and through a nail-facing side of nail body 210 and housing 221 to contact the nail bed of fingernail 8. As also shown in FIG. 7, bottom surface 213 of nail body 210 may comprise a flexible biocompatible material adapted to conform to any curvatures of nail plate 8 and the nail-contacting portion of haplic generator 240, such as an attachment element 216 operable to attach nail body 210 to and maintain an alignment of haptic generator 240 with fingernail 8. The nail-contacting portion of haptic energy generator 240 may extend through openings in bottom surface 213, housing 221, and attachment element 216 to contact fingernail 8. Isolation structure 241 and interior surfaces of the openings extending through bottom surface 213, housing 221, and/or attachment element 216 may comprise an energy-directing material operable to direct energies toward fingernail 8 through said openings, such as a metallic strip on the interior surfaces of the openings and/or ceramic particles contained within isolation structure 241 and/or attachment element 216.

Exemplary aspects of this disclosure are now described with reference to communication apparatus 300. As shown in FIGS. 3, 9, 10, and/or 11, communication apparatus 300 may comprise a nail body 310 and a data communication device 320 housed in the nail body 310.

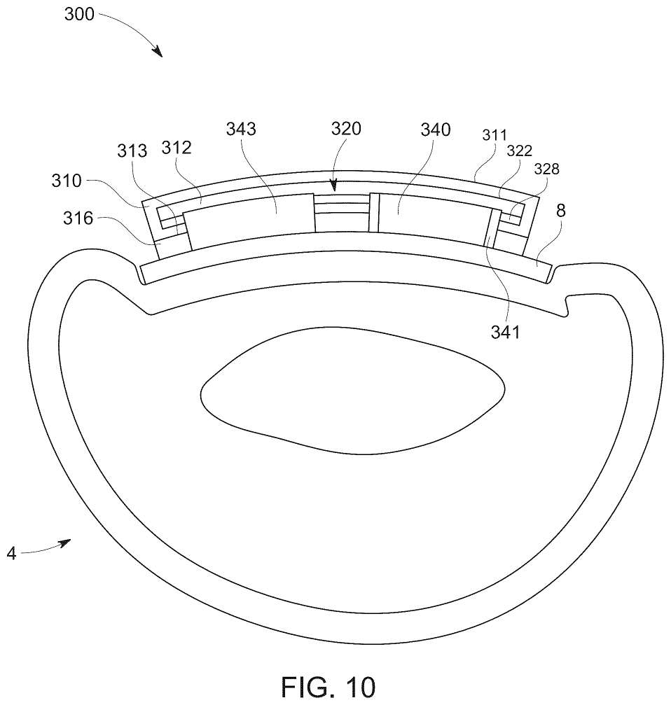

As shown in FIGS. 3, 9, and/or 11, nail body 310 may be similar or identical to nail body 110 and 210 described above, making it similarly mountable and wearable. As shown in FIGS. 9, 10, and/or 11, nail body 310 may comprise a top surface 311, an interior cavity 312, and a bottom surface 313 that are similar or identical to top surface 111, 211, interior cavity 112, 212, and bottom surface 113, 213; and interior cavity 312 may be similarly operable to house and facilitate interconnections between electronic components of data communication device 320, permit operation of its electronic components, and enable repeated uses of communication apparatus 300 with different users over an extend period of time.

In contrast to above, interior cavity 312 may be larger and/or comprise a plurality of different interior portions of nail body 310, in which each different portion is sized to house a different portion of data communication device 320. As shown in FIG. 10, interior cavity 312 may comprise two different interior portions of nail body 310 and a conduit extending therebetween to facilitate transfers of data and power.

Data communication device 320 may be operable to input control data from external device 11 over wireless network connection 9 and output one or more different energies to finger 4 responsive to the control data. Because of its position on the nail bed of fingernail 8 of finger 4, data communication device 320 may be uniquely configured to communicate with user 1's brain via mechanoreceptors and like nerves located in the tip of finger 4 at locations palmar of the nail bed.

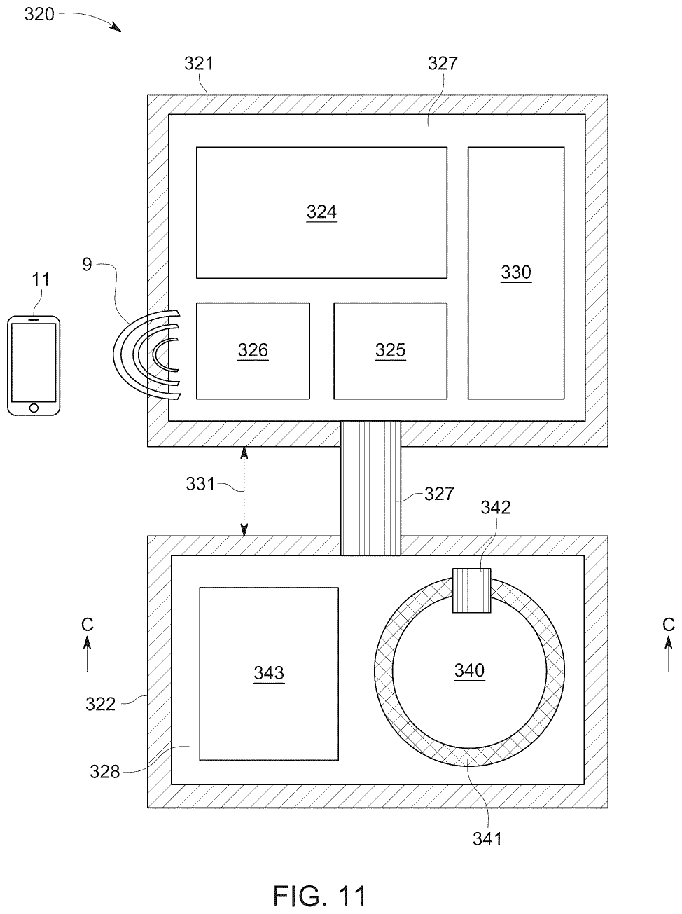

As shown in FIG. 11, data communication device 320 may comprise a first housing 321, a second housing 322, a power source 324, a controller 325, a data transceiver 326, a first PCB 327, second PCB 328, a first bus 329, a first haptic generator 340, an isolating structure 341, a second bus 342, and a second haptic generator 343. In contrast to above, first housing 321 may contain first PCB 327, second housing 322 may contain second PCB 328, and first bus 329 may extend between first PCB 327 and second PCB 328. First electronic components may be mounted to PCB 327 and operable to perform first operational functions for communication apparatus 300, such as such inputting data from external device 11, generating sensory data, and/or directing power to PCB 328. In complement, second electronic components may be mounted to PCB 328 and operable to perform second operational functions for apparatus 300, such as causing one or more different types of energy to be output toward fingernail 8 responsive to the first operational functions.

In keeping with above, nail body 310 may be formed with a molding method like described for nail body 110. First housing 321 may comprise a layer of polymeric material that is formed to contain the first electronic components; and second housing 322 may a separate layer of polymeric material that is formed to contain the first second components. Like housing 121 described above, first and second housings 321, 322 may similar prevent their respective first and second electronic components from being damaged by the molten biocompatible material during molding, and be operable with nail body 310 to hermetically seal those components within interior cavity 312.

As shown in FIG. 11, the first electronic components may comprise first PCB 327 and each of power source 324, controller 325, data transceiver 326, and sensor 330 mounted thereto; and the second electronic components may comprise second PCB 328 and each of first haptic generator 340 and second haptic generator 343 mounted thereto. Each of power source 324, controller 325, data transceiver 326, and sensor 330 may be similar or identical to their counterparts described above. First bus 329 may comprise a plurality of conductors extending between the first and second housings 321, 322 through to conduit of interior cavity 312, to transfer data and power between PCBs 327, 328.

First haptic generator 340 may comprise a first type of haptic energy generator operable to output a first type of haptic energy and second haptic generator 343 may comprise a second, different type of haptic energy generator operable to output a second, different type of haptic energy. As shown in FIG. 11, first haptic generator 340 a piezoelectric actuator operable to output a vibratory energy toward fingernail 8 and second haptic generator 343 may comprise an ultrasonic transducer or a thermoelectric generator operable to output an ultrasonic energy or a thermal energy toward fingernail 8. As shown in FIG. 11, first housing 321 may be spaced apart from second housing 322 by a gap 331 that is sized to minimize flows of energies from PCB 328 to PCB 327 when controller 325 is causing one or both of first haptic generator 340 and/or second haptic generator 343. Gap 331 may thus be described as a “first energy break” that is operable with PCB 328 to maximize the amount of energies output toward fingernail 8 from first haptic generator 340 and/or second haptic generator 343 and prevents those energies from being directly transferred to PCB 327. For example, because of gap 331, second housing 322 may be more effectively operable to transfer excess thermal energy to nail body 310 for subsequent transfer to ambient air surrounding finger 4, rather than to PCB 327 where it may otherwise affect the first operational functions performed therewith.

As shown in FIG. 11, like isolation structure 241, isolation structure 341 may be operable with first haptic generator 340 to direct flows of the first haptic energy toward fingernail 8 and limit flows of the first haptic energy in directions toward second haptic generator 343 and/or first PCB 327. Isolation structure 341 may thus be described as an “second energy break” that is operable with second PCB 328 to maximize flows of energies output toward fingernail 8 from first haptic generator 340 while simultaneously preventing those energies from negatively affecting the first electronic components mounted to first PCB 327. In keeping with above, isolation structure 341 may comprise a 0.50 to 1.50 mm thick layer of natural rubber, neoprene, and/or butyl rubber located between interior surfaces of hole extending through PCB 328 and exterior surfaces first haptic generator 340, such as a ring-like seal strip that wraps around haptic generator 340 and is mountable therewith.

As shown in FIGS. 10 and/or 11, nail-contacting portions of first haptic energy generator 340 and second haptic generator 343 may extend from interior cavity 312 and through a nail-facing side of nail body 310 and second housing 322 to contact the nail bed of fingernail 8. As also shown in FIGS. 10 and/or 11, bottom surface 313 an attachment element 316 operable to attach nail body 310 to and maintain an alignment of haptic generators 340, 343 with fingernail 8. The nail-contacting portions of haptic energy generators 340, 343 may extend through openings in bottom surface 313, housing 321, and attachment element 316 to contact fingernail 8. As above, isolation structure 341 and interior surfaces of the openings extending through bottom surface 313, second housing 322, and/or attachment element 316 may comprise an energy-directing material operable to direct energies toward fingernail 8 through said openings.

Exemplary aspects of this disclosure are now described with reference to communication apparatus 400. As shown in FIGS. 3, 12, 13, and/or 14, communication apparatus 400 may comprise a nail body 410, a data communication device 420 housed in the nail body 410, and a display element 450 mounted to nail body 410.

As shown in FIGS. 3, 12, and/or 13, nail body 410 may be similar or identical to nail body 110 and 210 described above, making it similarly mountable and wearable. As shown in FIGS. 12 and/or 13, nail body 410 may comprise a top surface 411, an interior cavity 412, and a bottom surface 413 that are similar or identical to top surface 111, 211 interior cavity 112, 212, and bottom surface 113, 213; and interior cavity 412 may be similarly operable to house and facilitate interconnections between electronic components of data communication device 420, permit operation of its electronic components, and enable repeated uses of communication apparatus 400 with different users over an extend period of time.

Data communication device 420 may be operable to input control data from external device 11 over wireless network connection 9 and output one or more different energies to finger 4 responsive to the control data. Because of its position on the nail bed of fingernail 8 of finger 4, data communication device 420 may be uniquely configured to communicate with user 1's brain via mechanoreceptors and like nerves located in the tip of finger 4 at locations palmar of the nail bed and/or via the eyes when display element 450 is positioned in their field of view by moving finger 4.

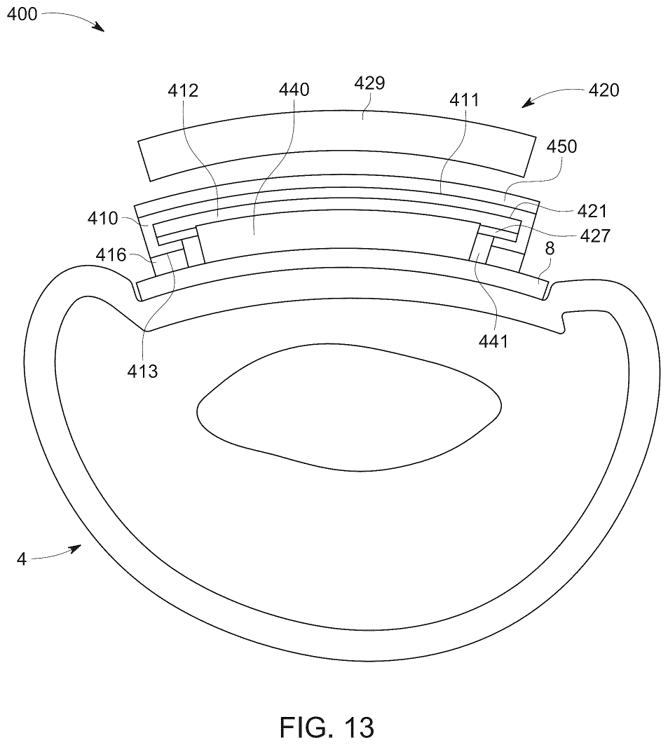

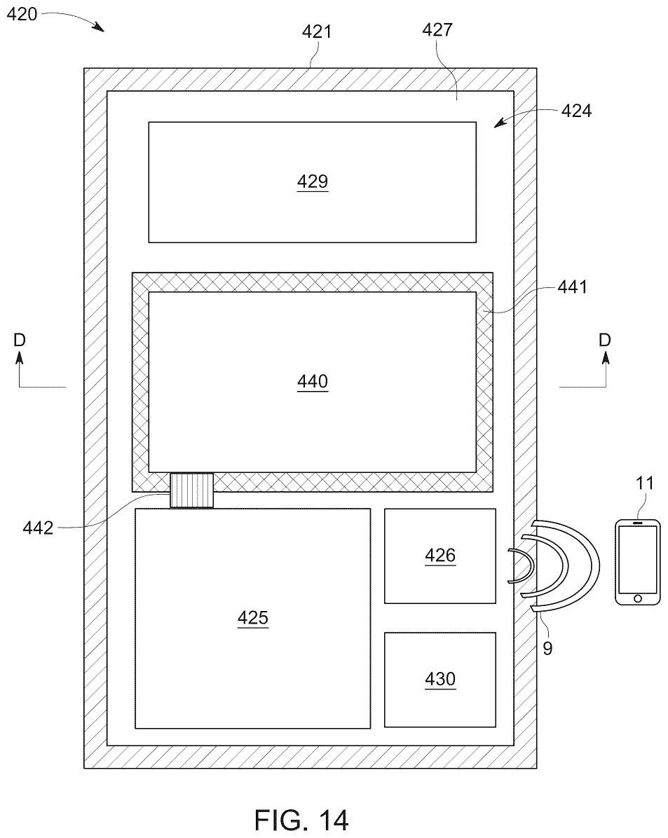

As shown in FIG. 14, data communication device 420 may comprise a housing 421, a power source 424, a controller 425, a data transceiver 426, a PCB 427, a bus 429, a haptic generator 440, and an isolating structure 441, each of which may be similar or identical to their counterparts described above for devices 120, 220, and/or 320, but for the differences now described. As shown in FIG. 14, power source 424 may include a removable battery 427 that can be charged or replaced without having to remove the entirety of communication apparatus 400 from fingernail 8. Removable battery 429 may comprise a lithium-ion battery with electrical conductors operable with corresponding conductors of a base portion of power source 424, a latch or quick release mechanism, and/or an electric coil for wireless charging. As shown FIG. 13, communication apparatus 400 may be mounted to a fingernail 8 using any an attachment element 416 that is similar or identical to attachment elements 116, 216, and/or 316 described above. As shown in FIG. 14, controller 425 may comprise haptic control circuits for haptic generator 440 and a video control circuits for display element 450. For example, controller 425 may be operable to receive control data from external device 11 and deliver power to haptic generator 440 via the haptic control circuits and/or the display element 450 via the video control circuits responsive to the control data.

Display element 450 may comprise any type of LCD or LED display technology, such as a MicroLED touchscreen panel like those sold by Samsung® for use with the Apple® Watch. As shown in FIG. 12, display element 450 may be mounted on top surface 413 of nail body 410 with an adhesive or similar means. For example, the video control circuitry of controller 425 may comprise an interface extending out of nail body 410 through top surface 413, an inner portion of a bottom surface of display element 450 may comprise a corresponding interface, and an outer portion of the bottom surface of display element 450 may be adhered to top surface 413 so that the interfaces are fused together. In this example, the interfaces may be completely surrounded by an adhesive so as to form a water-tight seal that prevents moisture from entering interior cavity 412.

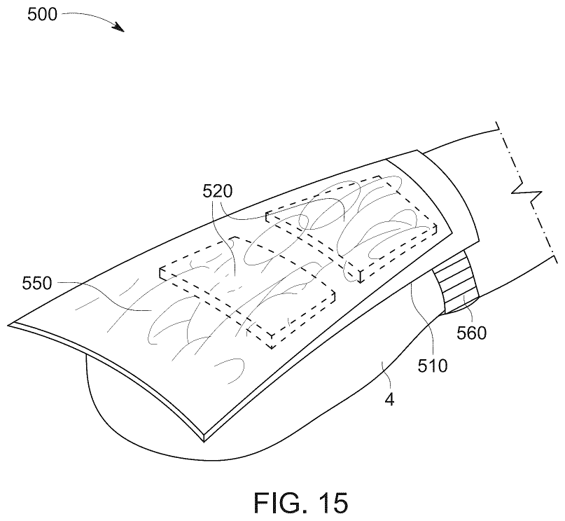

Exemplary aspects of this disclosure are now described with reference to communication apparatus 500. As shown in FIG. 15, communication apparatus 500 may comprise a nail body 510, a data communication device 520 housed in the nail body 510, and a display clement 550 mounted to nail body 510. Nail body 510, data communication device 510, and display element 550 may be similar or identical to their counterparts described above for device 420, but for the differences now described.

Display element 550 of communication apparatus 500 may be larger than display clement 450 of communication apparatus 400. As shown in FIG. 15, display element 550 may extend from at least a distal inter-phalangeal joint (DIP) of finger 4 to a location beyond the fingertip of finger 4. Nail body 510 may be extended and/or reinforce to accommodate the additional length of display element 550 and allow it to cantilever beyond the fingertip of finger 4. For example, the sides of nail body 510 may comprise thickened portions serving as beam elements providing additional flexural strength. To prevent communication apparatus 500 from falling during everyday use, communication apparatus 500 may include an attachment element 560 comprising a ring like structure that secures communication apparatus 500 to a distal portion of finger 4, such as the distal phalanx or proximal phalanx. Attachment element 560 may be fully adjustable allowing communication apparatus 500 to be mounted to any digit of the hand with or without utilizing a separate attachment element between apparatus 500 and fingernail 8. For example, to further maintain a position of nail body 510 on fingernail 8, a skin- and/or nail-contacting surface of nail body 510 may comprise an attachment element 516 comprising a biocompatible adhesive like those described above, providing additional attachment points for apparatus 500.

As shown in FIG. 1, communication device 1100 may be similar communication devices 100, 200, 300, 400, and/or 500, but with a nail body 1110 shaped to conform against a natural curvature of a toenail of a human toe digit, making it body-mountable and wearable on a nail plate of the toe digit in a manner similar to that of nail body 110, 210, 310, 410, and/or 510 on finger digit 4.

As shown in FIGS. 1, 2, 3, and/or 22, particular types of communication systems may be realized according to this disclosure with different combination of communication apparatuses 100, 200, 300, 400, 500, and/or 1100. Each communication apparatus 100, 200, 300, 400, 500, and/or 1100 may operate independently or in concert with each other and external device 11 within each type of communication system. Different type of data feedback loops may be established with each particular type of system. For example, sensory data collected from each communication apparatus 100, 200, 300, 400, 500, and/or 1100 may be communicated to a common same external device (e.g., AR system 21 of FIG. 2 worn by user 1) or different compatible external devices (e.g., another AR system 21 worn by user).

As shown in FIG. 2, certain advantages and performance benefits may be realized with a communication system 20 comprising: (A) at least one communication apparatus 100 with a computer readable top surface 111 (e.g., FIG. 5) on each hand 5 of user 1; (B) AR/VR system 21 comprising one or more downward-facing cameras 23 positioned to consistently capture and/or track each computer-readable top surface 111 during any augmented, virtual, and/or metaverse experience when user 1 is engaged with content output from AR/VR system 21; and/or (C) a pair of communication apparatus 1200 (e.g., described below and shown in FIG. 2), including a first apparatus 1224 wearable on user 1's left arm 3L and a second apparatus 1225 wearable on user 1's right arm 3R. By way of example, each communication apparatus, including apparatus 1224, 1225 may be similar to “apparatus 400” and its counterparts as described in International Patent Application No. PCT/US21/57634, filed Nov. 2, 2021, the entirety of which is hereby incorporated by reference into this application.

As shown in FIG. 3, certain advantages and performance benefits also may be realized with a communication system 40 comprising any number of communication apparatus 100, 200, 300, or 400, including one on each finger 4 of each hand. For example, communication system 40 may comprise: (A) one communication apparatus 100 operable to fixedly position a first machine-readable top surface 111 on the little finger of a left hand 5; (B) one communication apparatus 200 operable to fixedly position a second machine-readable top surface 211 on the ring finger of left hand 5; (C) one communication apparatus 300 operable to fixedly position a third machine-readable top surface 311 on the middle finger of left hand 5; (D) one communication apparatus 400 operable to fixedly position a fourth machine-readable top surface 411 on the pointer finger of left hand 5; and/or (E) another communication apparatus 100′ operable to fixedly position a fifth computer readable top surface 111 on the thumb of left hand 5.

Within communication system 40, communication apparatus 400 on the pointer finger and communication apparatus 100′ on the thumb may be a particularly useful combination because their machine-readable top surfaces 411, 111′ top surfaces are most often, easily, and naturally rotated upwards to toward the eyes of user 1 when their elbows and/or hands are lifted out in front while engaging the content output from AR/VR system 21. As shown in FIG. 3, display clement 450 of communication apparatus 400 on the point finger of left hand 5 may be operable to output a dynamic pattern to AR/VR system 21 and/or the eyes of user 1 during an augmented reality, IRL, mixed reality, virtual reality, and/or metaverse experience. Here for example, the natural rotation of the pointer finger and thumb during content engagement may help to align machine-readable top surfaces 411, 111 with the eyes of user 1 and/or downward facing cameras 23 during such experiences.

### Head- or Neck-Mounted Communication Apparatus, Methods, and Systems

Exemplary aspects of this disclosure are now described with reference to communication apparatus 600. Aspects of communication apparatus 600 may be like those of other communication apparatus described herein but within the 600 series of numbers. Any elements described herein with reference to another communication apparatus may be similarly described with references to apparatus 600. Certain differences are now described with reference to apparatus 600 and its unique capabilities within communication systems 10, 20, 30, and/or 40.

As shown in FIGS. 1 and 16-21, the anatomical positioning and energy-outputting capabilities of communication apparatus 600 make it uniquely suited for gathering particular types of physiological data from user 1's head and administering energy prescriptions by outputting different types of energy toward user 1's temple(s) responsive to the physiological data or an external data source. For example, because of its proximity to temporal arteries approximate to user 1's temple, such as the frontal branch of the superficial temporal artery, aspects of communication apparatus 600 may be operable to (i) measure and output brain and/or cardiovascular data by tracking physical changes associated with blood flowing through the temporal arteries; and (ii) prescriptively affect user 1's brain data by outputting electricity, light, and/or thermal energies toward the blood flowing through the temporal arteries. As a further example, because of its proximity to brain tissue underneath user 1's temple such as the temporal lobes, aspects of communication apparatus 600 also may be operable to (i) measure and output brain data by tracking changes associated with brain tissues proximate to the temples such as the temporal lobes, such as electrical signal changes associated with a brain activity of the frontal lobes, flow rate and/or temperature changes associated with blood flowing throw the frontal branch of the superficial temporal artery; and (ii) prescriptively affect the brain data by outputting electricity, light, and/or thermal energies toward the frontal lobes through the temple region and/or by outputting electrical and/or vibratory energies toward the temporalis muscle, the temporal nerve, and/or the Zygomaticofacial nerve.

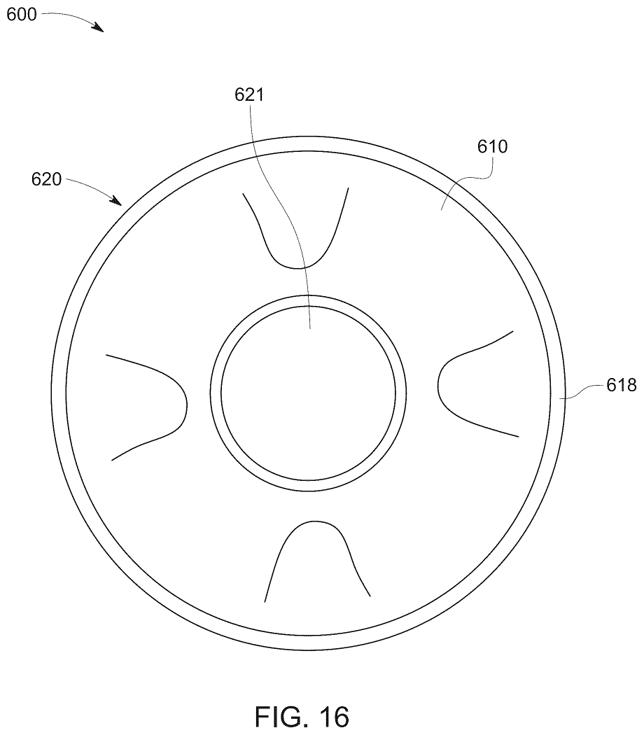

Additional aspects of communication apparatus 600 are now described. As shown in FIG. 16, communication apparatus 600 may comprise a housing 610 and a data communication device 620 housed in housing 610.

Housing 610 may be operable to attach data communication device 620 to the skin of user 1's temple and maintain an alignment of one or more elements of data communication device 620 with tissues underneath user 1's temple by acting against the skin, such as the temporal arteries and/or lobes. When attached to user 1's head with housing 610, communication apparatus 600 may thus be operable, with a processor (e.g., of apparatus 600, another apparatus described herein, and/or external device 11) to administer energy prescriptions by outputting different types of communicative and/or therapeutic energies toward user 1's temples responsive to a control signal output with the control application, such as the unified control signal.

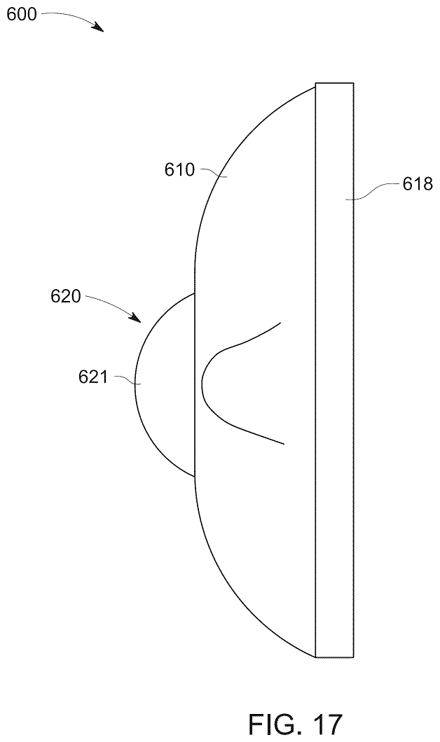

As shown in FIGS. 17 and/or 19, housing 610 may comprise a biocompatible material that fully or partially surrounds data communication device 620. For example, housing 610 may be 3D printed from a 3D printable material (e.g., a polymeric and/or metallic material) to have a skin-contacting surface shaped to obtain a custom fit with user 1's temple so that bio adhesive layer 618 may conform closely thereto, more effectively helping housing 610 remain attached thereto in diverse conditions. In this example, housing 610 and its skin-contacting surface may be shaped-to-fit based on a 3D scan, mold, picture, or other representation of user 1's temple. As shown in FIG. 1, because of its mass-customizable shape, the skin-contacting surface of housing 610 may both establish a precise fit with user 1's temple and generally maintain an alignment of data communication device 620 with the temple region and the tissues behind it.

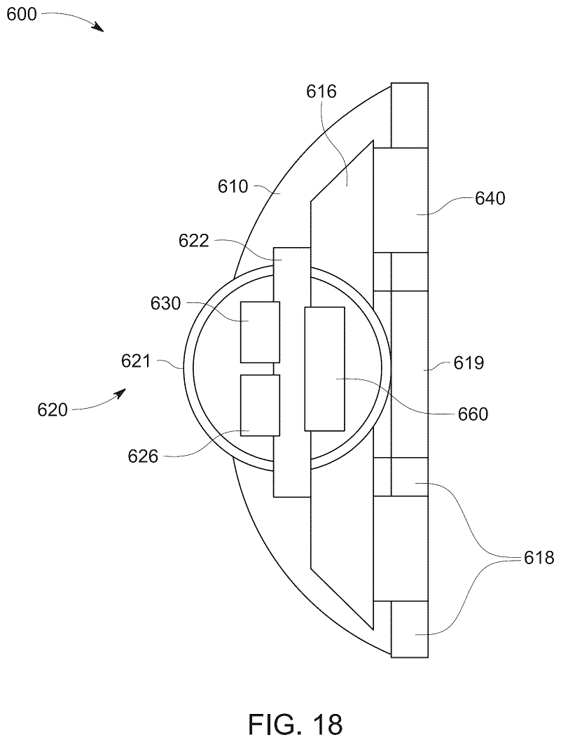

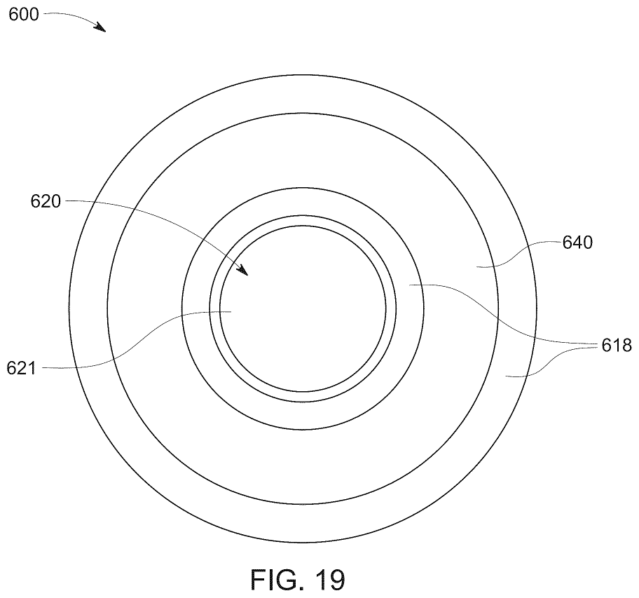

Housing 610 may comprise a bioadhesive layer 618 and an opening 619. As shown in FIG. 18, bioadhesive layer 618 may provide a convenient, biocompatible means for attaching communication apparatus 600 to user 1's temple as best shown in FIG. 1. Bioadhesive layer 618 also may be mass customizable. For example, a composition of biocompatible layer 618 may be formulated based on a dermatological profile of user 1's skin to promote irritation-free attachment for extended periods of time (e.g., hours). The composition of bioadhesive layer 618 may include any type of one- or two-part epoxy and silicone biocompatible adhesives, light-cured materials, epoxy-polyurethane blends, cyanoacrylates, and the like. Bioadhesive layer 618 may comprise one or more shapes positioned around components of data communication device 620. As shown in FIG. 19, bioadhesive layer 618 may comprise a plurality of annular shapes surrounding data communication device 620.

As shown in FIG. 18, opening 619 may extend through housing 610 and comprise different portions that are sized to allow elements of data communication device 620 to output light, thermal, vibrational and/or acoustic energies in directions toward user 1's temple, including an outward portion, a PCB receiving portion, and a skin-facing portion.

Housing 610 may act as a protective barrier. For example, housing 610 may be 3D printed from the 3D printable material to have a thickness that renders data communication device 620 effectively waterproof, even if exposed to water or worn in aquatic conditions. Bioadhesive layer 618 and/or other sealing elements may be used to seal housing 610. Bioadhesive layer 618 also may act as a protective barrier for the user. For example, because of its relative stiffness, layer 618 may absorb impact forces applied to housing 610 if struck by another object during movements of the user and/or have a tackiness that permits the skin-contacting surface of housing 610 to be removed from the skin by an accidently applied force without damaging the skin. As shown in FIG. 19, bioadhesive layer 618 also may direct flows of communicative and/or therapeutic energies in directions toward the skin and limit flows of those energies in directions along the skin, allowing the energies to be delivered with a higher efficiency and/or focused on discrete areas of the skin. For example, bioadhesive layer 618 may comprise energy directing elements (e.g., metallic elements or layers, like flecks, strips, or wraps of aluminum) that reflect most or nearly all light, vibration, and/or thermal energies toward the skin without affecting the tackiness of layer 618.

As shown in FIGS. 18, 20 and 21, data communication device 620 may comprise a shell 621, a printed circuit board or PCB 622, a power source 624, a controller 625, a data transceiver 626, a sensor 630, a thermoelectric generator 640, and an LED 660.

As shown in FIG. 18, shell 621 may comprise a semi-spherical shape defining an interior cavity that fully or partially surrounds these components of data communication device 620. Shell 621 may render data communication device 620 waterproof so that these components are protected against water damage from perspiration or elsewhere. As shown in FIG. 18, some electronic components of data communication device 620 may be hermetically sealed between shell 621 and PCB 622 whereas others may be located outside of shell 621. Like housing 610, shell 621 also may be 3D printed from a 3D printable material. As shown in FIGS. 16, 17, and/or 18, an outward-facing portion of shell 621 may be 3D printed from a radio translucent material and contained in the outward-facing portion of opening 619 so that an outward-facing surface of PCB 622 is positioned to send and receive data through the outward-facing portion of shell 621. As further shown in FIGS. 16, 17, and/or 18, a skin-facing portion of shell 621 may be 3D printed from an energy-translucent material and contained in the skin-facing portion of opening 619 so that a skin-facing surface of PCB 622 may be positioned to administer energy prescriptions by outputting a therapeutic energy toward user 1's temple and/or receiving a reflected portion of the therapeutic energy back from user 1's temple. As shown in FIGS. 18 and 19 and described further below, the skin-facing portion of shell 621 may be 3D printed from a light-transmitting materials to define a lens operable to diffuse and/or distribute light to and/or from user 1's temple.

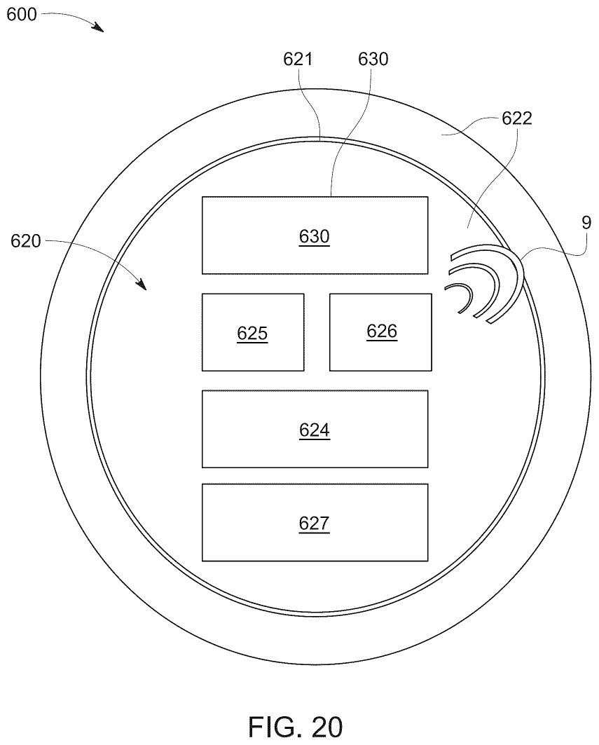

PCB 622 may be electrically and/or structurally engaged with each of housing 610, shell 621, power source 624, controller 625, data transceiver 626, sensor 630, and LED 660. In keeping with above, PCB 622 may comprise a structural frame including conductive tracks, pads and other features etched from one or more sheet layers of copper laminated onto and/or between sheet layers of a non-conductive substrate. As shown in FIGS. 18 and 20, PCB 622 may span between support surfaces of housing 610 and define network of conductors extending between power source 624, controller 625, data transceiver 626, sensor 630, and LED 660. As shown in FIGS. 18, 20, and/or 21, power source 624, controller 625, data transceiver 626, and sensor 630 may be mounted on an outward-facing side of PCB 622 and LED 660 may be mounted in a skin-facing side of PCB 622. As described above, the skin-facing and outward-facing portions of shell 621 may be optimized for use with power source 624, controller 625, data transceiver 626, and sensor 630 on one side of PCB 622 and LED 660 on the other side of PCB 622. For example, the skin-facing portion of shell 621 may be made of a first material (e.g., acrylic) and the outward-facing portions of shell 621 may be made of a second material (e.g., quartz).

The edges of PCB 622 may be contained in the PCB receiving portion of opening 619. As shown in FIG. 18, housing 610 may be 3D printed from a 3D printable material operable to diffuse heat and the edges of PCB 622 may be attached (e.g., adhered, screwed in, and/or soldered) to the PCB receiving portion of opening 619 with heat transferring connections operable to transfer heat from LED 660 into housing 610, allowing it to serve as a heat sink for LED 660. As shown in FIG. 20, PCB 622 may comprise a heat sink 627 that is thermally conducted to LED 660 and the heat transferring connections may comprise a first thermal conductor extending between heat sink 627 on the edges of PCB 622 and a second thermal conductor on interior surfaces of the PCB receiving portion of opening 619, in which the first and second thermal conductors are placed in physical contact with one another when PCB 622 is attached to housing 610. Because of the heat transferring connections described above, housing 610 may heat up after operating LED 660 for extended periods of time. In keeping with its protective nature, bioadhesive layer 618 may prevent housing 610 from transferring the heat directly to the skin so that the amount of heat output to user 1's temple may be more precisely controlled.

Power source 624 may comprise one or more battery elements, such as a lithium-ion battery pack and an inductive charger. In keeping with above, controller 625 and data transceiver 626 may comprise and/or be operable with any combination of technologies for receiving, processing, and/or sending data and control signals between communication apparatus 600 and another communication apparatus of communication data system 10. As shown in FIG. 20, controller 625 may comprise processing elements operable with data transceiver 626 to output data from sensor 630 to a computing device (e.g., like computing device 11 of FIG. 11, shown as an Apple® iPhone®) and/or receive control signals from the computing device over a wireless network 9 such as via Bluetooth or Wi-Fi.

Sensor 630 may be mounted to the outward-facing side of PCB 622 as shown in FIGS. 18 and 20 and/or located elsewhere on data communication device 620. Sensor 630 may comprise one or more electronic sensing elements that are powered by power source 624, in communication with controller 625 via conductors of PCB 622, and operable with controller 625 to capture different types of data about user 1, including audio data, movement data, physiological data, etc. As shown in FIG. 20, sensor 630 may comprise a camera, a microphone, a motion sensor, an inertial measurement unit, an optical sensor, and/or other electrically powered sensors.

Thermoelectric generator 640 may be like thermal generator element 142 of International PCT Patent App. No. US2021/030528, filed May 3, 2021, the entirety of which is hereby incorporated by reference into this application. For example, thermoelectric generator 640 also may communicate a thermal energy to the brain through nerves associated with the skin. As shown in FIGS. 18-20, thermoelectric generator 640 may convert electricity from power source 624 into an amount of thermal energy recognizable by temperature receptors of skin such as the Ruffini corpuscle as a hot thermal energy or a cold thermal energy. As shown in FIGS. 18 and 19, thermoelectric generator 640 may comprise a flexible thermoelectric generator having a rectangular cross-section and an outward-facing interface that is structurally attachable to housing 610 and electrically connectable to PCB 622.

As shown in FIGS. 18 and 19, the annular shape of thermoelectric generator 640 may define a continuous body with a wide tissue contact surface operable to evenly output one of a cold thermal energy or a hot thermal energy from the skin-facing surface of housing 610 in signal direction toward the skin. In many areas of the skin, for example, the density of Ruffini corpuscles may allow user 1 to experience the thermal energy as a continuous output despite there being a central opening extending through thermoelectric generator 640. As shown in FIGS. 18 and 19, thermoelectric generator 640 may comprise a flexible thermoelectric generator that is attached to the skin-facing surface of housing 610 at locations between bioadhesive layer 618 and electrical conductors extending back to data communication device 620, allowing for transmission of data and/or power therebetween. By way of example, thermoelectric generator 640 may comprise a flexible thermoelectric generator such as those sold by TEGway at www.tegway.co.

As shown in FIGS. 18 and/or 19, thermoelectric generator 640 may comprise an annular array of Peltier cooler semiconductors attached to one or more thermally conductive plates (e.g., aluminum plates) having an annular shape. The annular array of Peltier cooler semiconductors may be operable with power source 624 via controller 625 to create a temperature differential responsive to an electric current. The temperature differential may cause a skin-facing one of the annular plates to output one of hot or cold energy toward user 1's temple (e.g., heat) and an outward-facing one of the annular plates to output the other of hot or cold energy toward interior portions of housing 610. At a first, lower output level, the temperature differential may be perceivable by nerves associated with user 1's skin (e.g., by the Ruffini corpuscle) as different degrees and variable experiences of hot and cold. At a second, higher output level, the temperature differential may be utilized to heat or cool blood flowing to user 1's brain through temporal arteries. Rapid oscillations of thermal energy in the form of hot and cold cycles also may be realized, creating additional thermal effects, such as expanding and contracting the temporal arteries in a prescribed manner.

As shown in FIG. 18, housing 610 may comprise an air gap 616 located between shell 621 containing LED 660 and thermoelectric generator 640 to limit transfers of thermal energy between generator 640, PCB 622, and the other electronic components attached to PCB 622. As shown in FIG. 1 and described above, communication apparatus 600 may be attached to user 1's temples with bioadhesive layer 618 at a physical location that positions one or more portions of thermoelectric generator 640 over the temporal arteries of the user. Because of its position and capabilities, thermoelectric generator 640 may be operable at the first, higher output level to administer energy prescriptions for mental health by outputting thermal energies for communicating with user 1's brain via nerves associated with skin of the temple region; and at the second, higher output level to administer energy prescriptions for physical health by outputting thermal energies for transferring thermal energies into blood flowing through user 1's temporal arteries into the head and brain, thus providing a first means for affecting user 1's mental state with and a second means for affecting user 1's physical health.

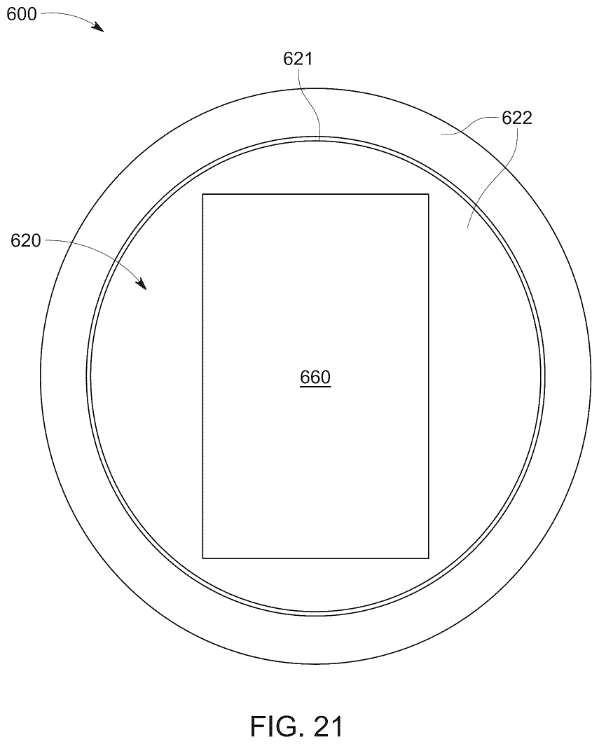

As shown in FIGS. 18 and 21, LED 660 may comprise a light emitting diode(s) that is mounted to a skin-facing side of PCB 622 and operable with controller 625 to output different types (e.g., wavelengths) of light directly to the user via opening 619. For example, LED 660 may comprise a multi-color LED, such as an RGB LED operable to output millions of different colors toward the user's temples at varying intensities. Opening 619 of housing 610 may be sized and positioned to allow LED 660 to output different types of light toward user 1's temple. As shown in FIG. 18, LED 660 may be mounted on the skin-facing surface of PCB 622 and positioned to output light through the skin-facing portion of shell 621, into opening 619, and toward user's 1 temple. The skin-facing portion of shell 621 may evenly distribute the light output from LED 660 to prevent hotspots or other concentrations of the light that may prove harmful. As shown in FIG. 18, the skin-facing portion of shell 621 may be 3D printed to define a semi-spherical lens that directs a particular wavelength of the light from LED 660 toward user 1's temporal arteries and/or guides a reflected portion of that light back toward an element of PCB 622 for further processing. Different wavelengths may be used. For example, LED 660 may be operable with power source 624 and controller 625 to output a red light for oxygenating the blood and/or a green light for measuring an oxygenation level of the blood. As shown in FIG. 18 and noted above, LED 660 may be spaced apart from thermoelectric generator 640 to create air gap 616 for the purpose of limiting transfers of thermal energy between LED 660 and thermoelectric generator 640.

As shown in FIGS. 18-20, LED 660 may be rendered waterproof by the skin-facing portion of shell 621, allowing data communication device 620 to remain on or adjacent the skin for extended periods of time without affecting LED 660. As also shown in FIG. 18, the skin-facing portion of shell 621 may comprise a translucent or semi-translucent lens structure with a semi-spherical shape that allows the different types of lights output from LED 660 to pass therethrough toward user 1's temple. The skin-facing portion of shell 621 may evenly distribute the different types of light to prevent hotspots or other concentrations that may prove harmful. In keeping with above, areas of the skin-facing portion of shell 621 may be 3D printed to comprise a lens portion operable to direct the different types of light from LED 660 toward user 1's temples. For example, LED 660 may output a red light toward user 1's temples to increase energy production at locations proximate to the brain by strengthening mitochondria of cells flowing through the temporal arteries and/or skin of user 1's temples and the lens portion of shell 621 may evenly distribute the red light. As a further example, LED 660 may be output a green light toward the temporal arteries and the lens portion of shell 621 may collect reflection portions of the green light toward an optical component of sensor 630. Because of its position and capabilities, LED 660 may administer energy prescriptions by delivering targeted applications of light-based therapy to nerves associated with the skin of user 1's temple and/or blood flowing through their temporal arteries into their head and brain, providing another means for affecting user 1's health.

In a therapeutic setting, sensor 630 may be operable with controller 625 to modify the therapeutic thermal energy output by thermoelectric generator 640 and/or the therapeutic light output by LED 660 responsive to data associated with user 1 or their environment. As shown in FIG. 1, sensor 630 may comprise one or more sensing elements, such as a light sensor (e.g., a photoelectric sensor) operable to adjust the light output by LED 660 responsive to a light level (e.g., lumens) of user 1's environment, a transducer (e.g., a microphone) operable to adjust the therapeutic thermal energy output by thermoelectric generator 640 responsive to an acoustic measure (e.g., dBs) of blood flowing through the temporal arteries or a sound of user 1's environment. In keeping with previous examples, controller 625 may be operable with an optical component of sensor 630 to determine cardiovascular measures associated with user 1, such as heart rate, blood pressure, and/or blood oxygenation, by causing LED 660 to output green light toward the temporal arteries, receiving reflected portions of the green light with the optical component of sensor 630 (e.g., through the lens portion of shell 621), and predicting the cardiovascular measures based on an analysis of the reflected portions. As a further example, controller 625 also may be operable with the optical component of sensor 630 to determine the cardiovascular measures by causing thermoelectric generator 640 to output a cold thermal energy toward the temporal arteries while outputting the green light from LED 660, using the reflected portions of the light to quantify physical changes of the temporal arteries (e.g., estimated changes in diameter) responsive to the cold thermal energy, and predicting the cardiovascular measures based on the quantified physical changes.

As shown in FIG. 22 and noted above, for example, certain advantages and performance benefits also may be realized with a communication system 30 comprising: (A) a communication apparatus 100, 400 with LCD screen 442 (e.g., FIG. 9) on one digit of hand 5 of user 1; and (B) a communication apparatus 600 (e.g., FIGS. 16-21) mounted on user 1's head and/or neck region, such as above or proximate to their jugular vein. As shown in FIGS. 4 and 14, for example, data transceiver 426 may be operable to receive GPS data from external device 11 or sensor 430; controller 425 may be operable to cause LCD screen 442 to output a directional graphic (e.g., a compass arrow) responsive to the GPS data; haptic generator 440 may comprise one or more piezoelectric vibrating elements operable to output vibratory energies to user 1's fingertip if/when the directional graphic is aligned with an intended direction of travel; and sensor 430 may be operable to capture physiological data associated with user 1 while traveling. As shown in FIGS. 1, 16, and 22, for example, annular thermoelectric generator 640 also may respond to the aforementioned GPS data to indicate whether user 1 is traveling at a desired rate of speed, in which a small amount of heat tells user 1 to move faster and a small amount of cold energy tells user 1 to slow down, like a classic game of “hot and cold”. In these examples, sensor 630 may utilized to determination an oxygenation level of user 1's blood one or both of thermoelectric generator 640 and LED 660 may be operable with controller 625 to automatically apply prescribed combinations of light and/or thermal energies to increase blood oxygenation levels.

As shown in FIG. 1, communication system 10 may comprise a first communication apparatus 600 adhered to skin of a medial temple region of user 1 and a second communication apparatus 600′ adhered to skin of a lateral temple region of user 1. Because they are positioned on opposite sides of user 1's brain when worm, first communication apparatus 600 may be operable with second communication apparatus 600′ within communication system 10 to (i) measure and output brain data by outputting first energies (e.g., optical, thermal, and/or ultrasonic energies) from communication apparatus 600 laterally and/or outputting second energies (e.g., optical, thermal, and/or ultrasonic energies) from communication apparatus 600′ medially, receiving reflected portions of the first and/or second energies, and determining changes associated with brain tissues proximate to the temples such as the temporal lobes based on the reflected portions, such as electrical signal changes associated with a brain activity of the frontal lobes, flow rate and/or temperature changes associated with blood flowing through the frontal branch of the superficial temporal artery; and (ii) prescriptively affect the brain data by outputting the first and second energies to include electricity, light, and/or thermal energies directed toward the frontal lobes through the temple region and/or by outputting electrical and/or vibratory energies toward the temporalis muscle, the temporal nerve, and/or the Zygomaticofacial nerve.

Although described as a standalone wearable, it is contemplated that aspects of communication apparatus 600 and/or 600′ may be built into another wearable device suitable for placement of against user 1's temples. As shown in FIG. 2, the electronic components of communication apparatus 600 may be integrated into a lateral side of AV/VR system 21 and the electronic components of communication apparatus 600′ may be integrated into a lateral side of AV/VR system 21, thereby allowing apparatus 600, 600′ to be maintained against user 1's temples with or without the use of biocompatible adhesives and separate power sources.

### Body-Mounted and/or Wearable Communication Apparatus

Exemplary aspects of this disclosure are now described with reference to communication apparatus 700. Aspects of communication apparatus 700 may be like those of other communication apparatus described herein but within the 700 series of numbers. Any elements described herein with reference to another communication apparatus may be similarly described with references to apparatus 700. Certain differences are now described with reference to apparatus 700 and its unique capabilities within communication systems 10, 20, 30, and/or 40.

As shown in FIGS. 1 and 23-26, the anatomical positioning and energy-outputting capabilities of communication apparatus 700 make it uniquely suited for gathering particular types of physiological data from user 1's chest and administering energy prescriptions by causing a plurality of energy generators to output one or more different energy types toward a plurality of different areas of user 1's skin responsive to the physiological data or an external data source. For example, because of its proximity to user 1's heart, lungs, and other internal organs, aspects of communication apparatus 700 may be operable to (i) measure and output physiological data associated with user 1's chest by tracking physical changes associated with blood flowing through the heart and air flowing through the lungs; (ii) gather and output additional physiological data from other locations on user 1's body, such as from the fingertips with an array of communication apparatus 100, 300, and/or 400 on hand 5 and the head with an array of data communication apparatus 600 on the temples and/or neck; and (iii) prescriptively affect user 1's physiological data by causing haptic generator 240, haptic generator 340, haptic generator 343, haptic generator 440, the haptic generator of communication apparatus 500, thermoelectric generator 640, LED 660, and/or any counterpart generating elements described herein to output one or more different types of energy to a plurality of different locations on user 1's body in a coordinated manner responsive to the physiological data measured and output with communication apparatus 700.

Communication apparatus 700 may thus serve as a central control element or “master controller” for communication system 10. For example, each controller of each communication apparatus 100, 200, 300, 400, 500, 600, 700, 800, 900, 1000, 1100, 1200 and/or 1300 may be in data communication with controller 725 of communication apparatus 700, which itself may be operable to (i) communicate a set of physiological data generated by sensing elements of communication apparatus 100, 300, 400, 600, and/or 700 to external device 11 over wireless data 9; (ii) receive the unified control signal from device 11; and (iii) distribute the control signals to 100, 200, 300, 400, 500, 600, etc. according to the unified control signal, causing their respective controllers and power sources to administer an energy prescription to the plurality of different locations on user 1's body.

External device 11 also may serve as the master controller. For example, external device 11 may comprise a cloud computing system and each controller of each communication apparatus 100, 200, 300, 400, 500, 600, 700, 800, 900, 1000, 1100, 1200 and/or 1300 may be in data communication with external device 11, which itself may be operable to (i) receive the set of physiological data via wireless data 9; (ii) generate the unified control signal from device 11; and (iii) cause one or more of the controllers distribute the unified control signal to the other controllers of each communication apparatus 100, 200, 300, 400, 500, 600, 700, 800, 900, 1000, 1100, 1200, and/or 1300, causing their respective controllers and power sources to administer an energy prescription to the plurality of different locations on user 1's body.

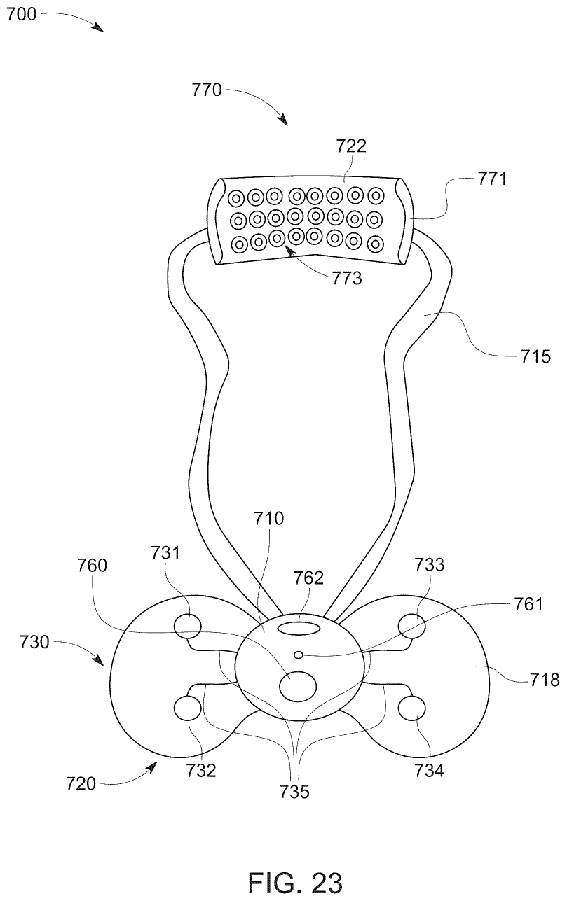

Additional aspects of communication apparatus 700 are now described. As shown in FIG. 23, communication apparatus 700 may comprise a housing 710 and a data communication device 720 housed in housing 710.

Housing 710 may be operable to attach data communication device 720 to the skin of user 1's chest and maintain an alignment of data communication device 720 with user 1's chest, heart, lungs, and/or head by acting against the skin. Housing 710 may comprise a biocompatible structure that renders data communication device 720 waterproof so that its components may be protected against water damage from perspiration or elsewhere. As shown at left in FIG. 23, housing 710 may be manufactured from a flowable silicon material (e.g., liquid silicone rubber) that surrounds and electrically insulates the electrically operable components of data communication device 720 and provides them with a degree of anti-shock protection by absorbing impact energies. For example, data communication device 720 may be placed in mold of any shape; the flowable silicon material may be input to the mold, surrounding data communication device 720; and the mold may be heated to form housing 710 into its final form by hardening the silicone material. Although shown as having an orb shape in FIG. 23, it is contemplated that housing 710 may be manufactured to assume any form using this method, such as heart shaped form. The electronic components of data communication device 720 may be hermetically sealed in housing 710 by the flowable silicone material in this manner, making it durable and waterproof.

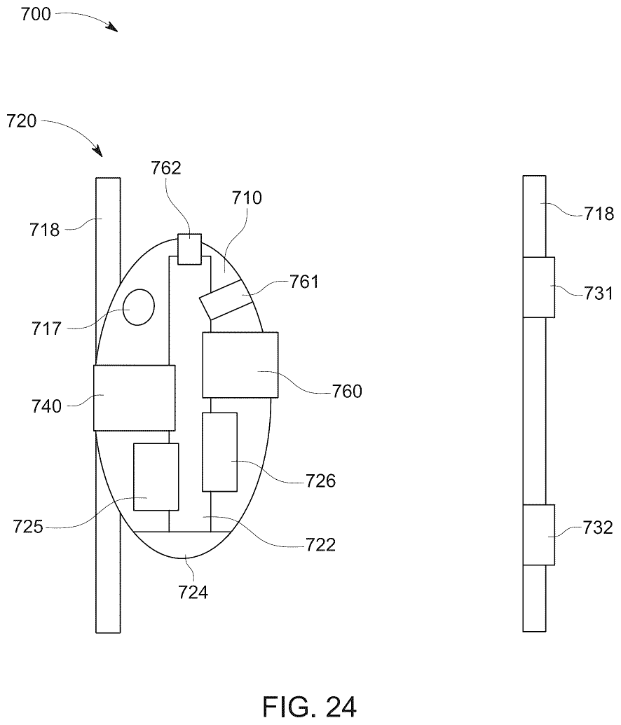

Communication apparatus 700 may comprise an attachment plate 718 operable to attach data communication device 720 to user 1's chest. As shown in FIGS. 23 and 24, attachment plate 718 may be a semi-rigid body extending outwardly from each data communication device 720 to define a skin-facing surface area for attachment user 1's skin. In keeping with above, the skin-facing surface area of plate 718 may comprise a biocompatible adhesive layer so that communication apparatus 700 may worn by pressing the skin-facing surface area of attachment plate 718 into user 1's chest, bending attachment plate 718 to fit counters of the chest, and allowing the biocompatible adhesive layer to obtain an adhesive bond with user 1's skin. The additional rigidity and larger skin-facing surface area of attachment plate 718 may allow the adhesive bond to readily support the vertical weight of data communication device 720 and/or any moment forces applied by device 720 to attachment plate 718 during normal movements of user 1. Thickness of attachment plate 718 may prevent it from tearing when applied to or removed from user 1's chest.

As with other bioadhesive elements described herein, the biocompatible adhesive layer of attachment plate 718 may provide a convenient, biocompatible means for attaching communication apparatus 700 to user 1's skin. In keeping with above, the biocompatible adhesive may be mass customizable based on a dermatological profile of user 1 to promote irritation-free attachment to user 1's chest for extended periods of time (e.g., hours) and/or comprise any type of one- or two-part epoxy and silicone biocompatible adhesives, light-cured materials, epoxy-polyurethane blends, cyanoacrylates, and the like. Other aspects of attachment plate 718 also may be customized. As shown in FIG. 1, attachment plate 718 and its biocompatible adhesive layer may be translucent so that communication apparatus 700 appears to float on the chest and/or comprise a printed graphic so that apparatus 700 appears to be part of tattoo.

As shown in FIGS. 23, 24, 25, and/or 26, data communication device 720 may comprise a PCB 722, a power source 724, a controller 725, a data transceiver 726, a sensor array (or multimodal sensor array) 730, and a bus 740.

PCB 722 may be contained in housing 711. In keeping with above, PCB 722 may comprise a structural frame including conductive tracks, pads and other features etched from one or more sheet layers of copper laminated onto and/or between sheet layers of a non-conductive substrate. As shown in FIG. 24, power source 724, controller 725, data transceiver 726, sensor array 730, and bus 740 may be structurally and/or electrically connected to PCB 722.

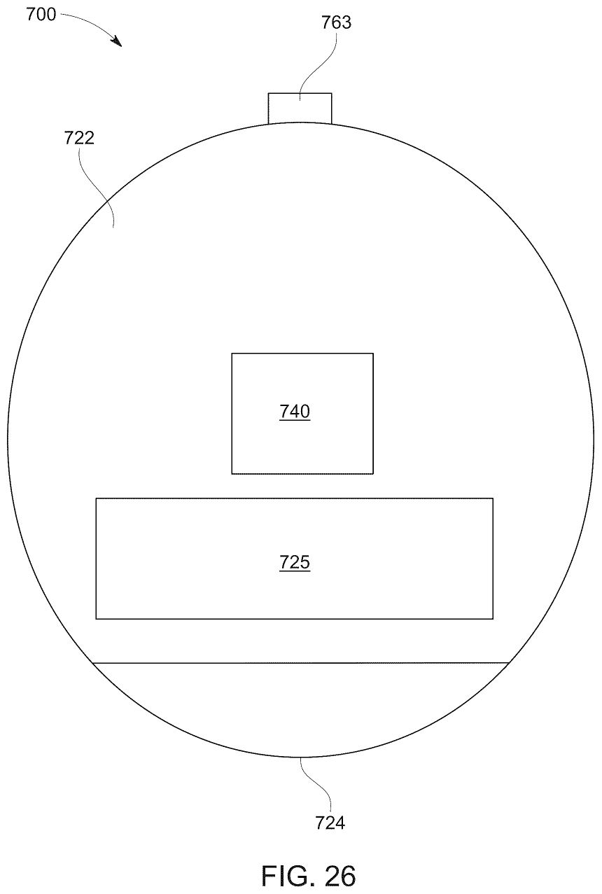

Power source 724 may comprise one or more battery elements, such as a lithium-ion battery pack and an inductive charger. As shown in FIGS. 24, 25, and 26 power source 724 may be located at one end of PCB 722 and exposed from housing 710. Controller 725 and data transceiver 726 may comprise and/or be operable with any combination of technologies for receiving, processing, and/or sending data and control signals between communication apparatus 700 and another communication apparatus of communication data system 10. As shown in FIG. 26, controller 725 may comprise processing elements operable with sensor array 730 and data transceiver 726 to measure and output physiological data to external device 11 and/or receive the unified control signal from external device 11 over wireless network 9.

Sensor array 730 may comprise a plurality of different sensing elements operable to generate physiological data associated with user 1's chest, heat, lungs, and/or head. As shown in FIGS. 23, 24, 25, and/or 26, sensor array 730 may comprise skin-mounted sensors 731-734, a forward-facing camera 760, a microphone 761, and an upward facing camera 762.

As shown in FIG. 23, skin-mounted sensors 731-734 may be positioned on or adjacent skin of user 1's chest and operable to output physiological data associated with user 1's heart and lungs. For example, sensor 731 may comprise a first microphone positioned for generating first audio data (i.e., by “listening”) associated with user 1's right lung; sensor 732 may comprise a second microphone positioned for generating second audio data associated to with the right side of user 1's heart; sensor 733 may comprise a third microphone positioned for generating third audio data associated with user 1's left lung; and sensor 734 may comprise fourth microphone positioned for generating fourth audio data associated to with the left side of user 1's heart. Different types of sensors may be similarly used. According to this disclosure, sensors 731-734 may comprise electrodes, other types of transducers, and/or any other sensing elements that are similarly operable to generate sensory data associated with both sides of the lungs and heart.

As shown in FIGS. 23, 24, 25, and 26, forward-facing camera 760 may be pointed outwardly from user 1's chest when data communication device 720 is attached thereto with attachment plate 718. Forward-facing camera 760 may be operable with controller 725 and/or external device 11 to generate first contextual data associated with user 1. For example, a firmware for forward-facing camera 760 may comprise a first AI-powered codec operable with controller 725 and/or a processor in data communication therewith to identify any faces, signs, and/or objects positioned in front of user 1's chest. Forward-facing camera 760 may thus be operable to output first contextual data associated with a first perspective of user 1's experience. Upward-facing camera 762 may be pointed upwardly toward user 1's face and operable to generate second contextual data associated therewith. For example, a firmware for upward-facing camera 762 may comprise a second AI-powered codec operable with controller 725 and/or a processor in data communication therewith to predict user 1's emotional state at a given time by comparing a partial view of user 1's face at the time with other images of user 1's face that have been previously associated with certain emotions and added to a training set for the second AI. Upward-facing camera 762 may thus be operable to output second contextual data associated with a second perspective of user 1's experience.

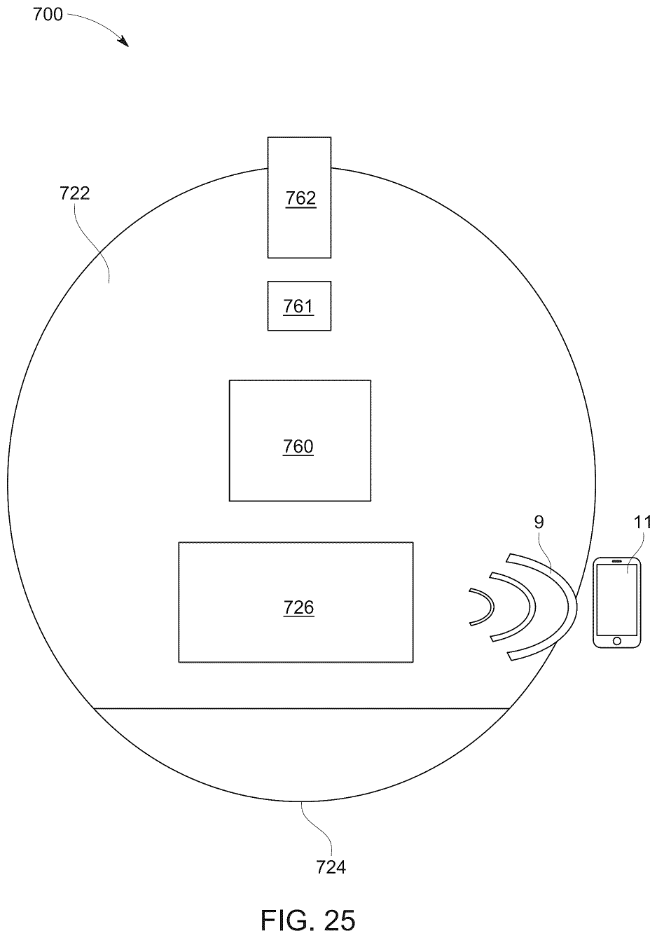

As shown in FIGS. 24 and 25, microphone 761 may be located either partially or fully within housing 710 and operable with controller 725 and/or a processor in data communication therewith to capture and transmit sound adjacent user 1. Microphone 761 may comprise a fifth microphone positioned for generating fifth audio data associated with the first and/or second perspectives of user 1's experience with system 10. Although shown as a standalone element, microphone 761 also may be incorporated into another element of sensor array 730 and/or comprise a plurality of audio recording elements. Microphone 761 may be operable with forward-facing camera 760 and/or upward-facing camera 762 to generate additional contextual data about user 1. For example, a firmware for microphone 761 may comprise a third AI-powered codec operable with controller 725 and/or a processor in data communication therewith to identify people or objects in front of user 1 by comparing the recording data with recordings that have been previously associated with certain persons, surroundings, or events and added to a training set for the third AI. For example, the training set also may comprise a database of identified profiles for external sounds and/or techniques, allowing the third AI to predict user 1's emotional state without the benefit of video data.

Attachment plate 718 may be operable to maintain positions of data communication device 720 and skin-mounted sensors 731-734 on user 1's chest. As shown in FIG. 24, each of sensors 731-734 may be attached to, embedded in, or otherwise supported by attachment plate 718 and connected to PCB 722 by flexible conductors 732 that also are attached to, embedded in, or otherwise supported by attachment plate 718. As shown in FIG. 23 and described herein, attachment plate 718 may be operable to maintain positions of sensor 731 relative to user 1's right lung, sensor 732 relative to the right side of user 1's heart, sensor 733 relative to user 1's left lung, and sensor 734 relative to the left side of user 1's heart. Housing 710 may be attached to attachment plate 718 with a partial moment connection that allows for the transfer of bending moment forces therebetween to generally maintain an alignment of forward-facing camera 760 relative to ground level and/or an alignment of upward-facing camera 762 relative to user 1's face. The rigidity, larger skin-facing surface area, and thickness of attachment plate 718 may allow its adhesive bond with user 1's skin to resist the bending moment forces during normal movements of user 1. As shown in at left FIG. 24, because of this connection, upward-facing camera 762 may be spaced apart from user 1's chest when attachment plate 718 is attached to user 1's chest so that camera 762 is better positioned to obtain partial views of user 1's face. In this configuration, data from upward-facing camera 762 and forward-facing camera 760 may be more effectively stitched together to collectively provide a wide-angle perspective of user 1's experience.

Communication bus 740 may be operable to transfer power to sensor array 730 from power source 724 and transfer physiological data generated with sensor array 730 to controller 725 for distribute to external device 11. As shown in FIGS. 24 and 25, bus 740 may comprise a flexible bundle of conductors extending out of attachment plate 718 and into housing 710 for conductive attachment to sensor array 730, power source 724, and controller 725.

As shown in FIG. 23, communication apparatus 700 may comprise an optional lanyard 715 that is operable together with or as an alternative to attachment plate 718. As shown in FIG. 23, optional lanyard 715 may comprise a neck support structure 771, a tissue interface 772, and a plurality of haptic energy generators 773. Neck support structure 771 may be placed against skin on the back of user 1's neck and operable to support the weight of communication apparatus 700 at least partially from user 1's neck. For example, neck support structure 771 may be operable with attachment plate 718 to maintain an alignment of forward-facing camera 760 relative to ground level and/or an alignment of upward-facing camera 762 relative to user 1's face. Tissue interface 722 may comprise a biocompatible surface of neck support structure 771, such as a layer of medical grade silicon. As shown in FIG. 23, tissue interface 722 also may comprise a bioadhesive silicon-based tape with a tackiness that allows for attachment to user 1's neck.

Plurality of haptic energy generators 773 may comprise an array of single- or multi-energy haptic energy generators operable with controller 725 to output difference types and/or combinations of haptic energies toward nerves associated with the skin on the back of user 1's neck. For example, each haptic energy generator 773 may comprise a multi-energy haptic generator like those described above. As shown in FIG. 23, lanyard 715 may comprise a structural sheath operable to transfer forces between neck support 771 and housing 710 and internal wiring operable to transfer data and power between plurality of haptic energy generators 773, power source 724, and/or controller 725.

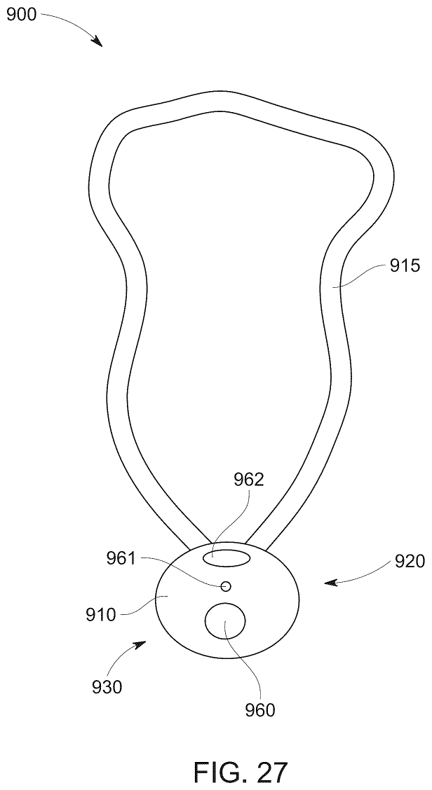

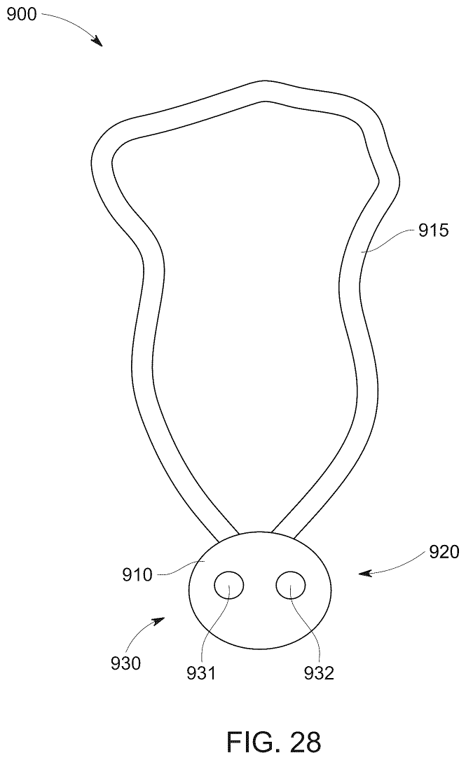

Exemplary aspects of communication apparatus 900 are now described. As shown in FIG. 27, communication apparatus 900 may be described as a simplified version of communication apparatus 700 with comparable sensing capabilities. Rather than being adhered to user 1's, communication apparatus 900 may be suspended from user 1's neck and positioned over the chest like an amulet adjacent user 1's heart and lungs. As shown in FIGS. 27 and 28, communication apparatus 900 may comprise a housing 910, a data communication device 920, and a lanyard 915, examples of which are now described.

Housing 910 may have a shape like that of housing 710 but without its connection to attachment plate 718, which may be omitted from communication apparatus 900 as shown in FIGS. 27 and 28. Housing 910 may be made of a material that is heavier than that of housing 710 and thus easier to position over the chest and maintain that position over time. Housing 910 may comprise a wearable body operable to generally maintain a position of data communication device 920 over user 1's chest and lungs when apparatus 900 is worn on user 1. Aspects of housing 910 may be like “wearable body 533” of International PCT Patent App. No. US2021/030528, filed May 3, 2021, the entirety of which is hereby incorporated by reference into this application. For example, housing 910 may comprise a decorative material having established value and/or associated affective properties, such as a crystalline structure and/or a polymeric structure, such as a rose quartz crystal structure. As shown in FIGS. 27 and 28, housing 910 may comprise an upper portion adapted to support communication apparatus 900 from the neck of user 1 so that a skin-facing surface of housing 910 may be maintained over the chest of user 1 by gravity forces.

As shown in FIGS. 27 and 28, housing 910 may comprise an interior cavity and data communication device 920 may be mounted in the interior cavity. Portions of housing 910 may conduct and/or transfer one or more different haptic energies output by data communication device 920. For example, the crystalline and/or a polymeric structure of housing 910 may store thermal energies output from a thermoelectric generator of data communication device 920 (e.g., like thermoelectric generator 640) and transfer vibratory energies from a piezoelectric or linear resonate actuator of device 920 (e.g., haptic generator 240). As shown in FIG. 28, the skin-facing surface of housing 910 may comprise a surface feature operable with user 1's garment to maintain a position of communication apparatus 900 over user 1's chest and heart. For example, the surface feature may comprise a Velcro® strip that is removably attachable to a corresponding Velcro® strip on user 1's shirt. As a further example, user 1's shirt may comprise a compression shirt (e.g., from Under Armour®) with a grommet and the surface feature may comprise a rim operable with the grommet to attach communication apparatus 900 to the compression shirt so that is elastic portions may be operable to maintain communication apparatus 900 over user 1's chest and heart.

As shown inn FIGS. 27 and 28, data communication device 920 may comprise a printed circuit board or PCB, a power source, a controller, a data transceiver, a sensor array, and a bus like those of apparatus 700 described above. As shown, the sensor array of data communication device 920 may similarly comprise a forward-facing camera 960, a microphone 961, and an upward-facing camera 962 like those of apparatus 700. As shown in FIG. 28, for example, the sensor array of data communication device 920 also may comprise a first skin-facing sensor 931 comprising a first microphone, electrode(s), and/or optical sensor positioned for generating first physiological data (i.e., by “listening”) associated with user 1's right lung and/or the right side of their heart and a second skin-facing sensor 932 may comprise a second microphone, electrode(s), and/or optical sensor positioned for generating second physiological data associated with user 1's left lung and/or the left side of their heart.

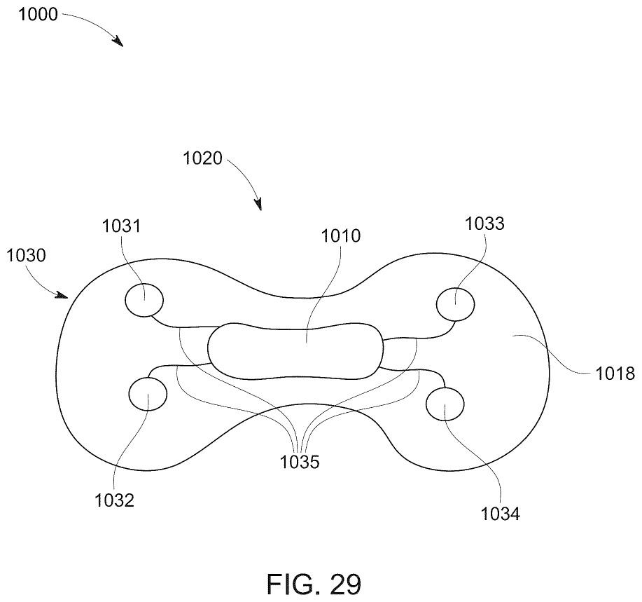

Lanyard 915 may be attached to data communication device 920 so that user may wear communication apparatus 900 around their neck without the need for adhesive attachment to user 1's body. As shown in FIGS. 27 and 28, lanyard 915 may be like optional lanyard 715 described above. For example, lanyard 915 may comprise structures for supporting communication apparatus 700 from user 1's neck and maintaining alignments of sensor array 930. As shown in FIG. 29, the skin-facing side of housing 910 may be curved to help maintain the position of apparatus 900 on user 1's chest and position skin-mounted sensors 931 and 932 against user 1's skin. Although not required, lanyard 915 also may contain various electronic components of apparatus 900, such as an elongated power source offering extended battery times or a linear array of single- or multi-energy haptic generators like generators 773.

Exemplary aspects of communication apparatus 1000 are now described. As shown in FIG. 29, communication apparatus 1000 may be a further simplified version of communication apparatus 700 and 800 comprising a housing 1010, a data communication device 1020, and an attachment plate 1018. Housing 1010 may be like housing 710 but designed for use underneath user 1's clothing, meaning that it may not include cameras. As shown in FIG. 29, housing 1010 may be operable to contain and house elements of data communication device 1020, such as a power source and a controller. Like housing 121 of communication apparatus 100, housing 1010 may hermetically seal those elements so that apparatus 1000 may be durable enough for everyday use. Sidewalls of housing 1010 may allow data and power to pass therethrough. For example, housing 1010 may comprise a radio-translucent polymeric material that allows Bluetooth signals and electricity to flow wireless therethrough so that electrical components of data communication device 1020 may be operable and charged through housing 1010.

Data communication device 1020 may comprise a printed circuit board or PCB, a power source, a controller, a data transceiver, a sensor array 1030, and a bus like those of apparatus 700 and 800. As shown in FIG. 29, the sensor array of data communication device 1020 may be positioned on or adjacent skin of user 1's chest and operable to output physiological data associated with user 1's heart and lungs. As shown in FIG. 29, the sensor array of device 1020 may comprise: a first skin-mounted sensor 1031 comprising a first microphone, electrode(s), and/or optical sensor positioned for generating first physiological data (i.e., by “listening”) associated with user 1's right lung; a second skin-mounted sensor 1032 comprising a second microphone, electrode(s), and/or optical sensor positioned for generating second physiological data associated with the right side of user 1's heart; a third skin-mounted sensor 1033 comprising a third microphone, electrode(s), and/or optical sensor positioned for generating third physiological data associated with user 1's left lung; and a fourth skin-mounted sensor 1034 comprising a fourth microphone, electrode(s), and/or optical sensor positioned for generating a fourth physiological data set associated with the left side of user 1's heart. According to this disclosure, sensors 1031-1034 may comprise electrodes, other types of transducers, and/or any other sensing elements that are similarly operable to generate sensory data associated with both sides of the lungs and heart.

Attachment plate 1018 may be similar or identical attachment plate 718. As shown in FIG. 29, attachment plate 1018 may similarly comprise a bioadhesive adhesive layer (e.g., a tape) with a tackiness that allows for attachment of apparatus 1000 to user 1's chest as best shown in FIG. 1, a thickness that prevents plate 1018 from tearing when applied to or removed from user 1's chest, and a degree of water-resistance that allows data communication device 1020 to be worn under user 1's shirt during more vigorous activity and/or while user 1 is partially or fully submerged in water. As shown in FIG. 29, attachment plate 1018 may be similarly operable to maintain positions of data communication device 1020 and skin-mounted sensors 1031-1034 on user 1's chest. For example, each of sensors 1031-1034 may be attached to, embedded in, or otherwise supported by attachment plate 1018 and connected to the PCB of data communication device 1020 with flexible conductors 1035 that also are attached to, embedded in, or otherwise supported by attachment plate 1018. As shown in FIG. 29 and described herein, attachment plate 1018 also may be operable to maintain positions of sensor 1031 relative to user 1's right lung, sensor 1032 relative to the right side of user 1's heart, sensor 1033 relative to user 1's left lung, and sensor 1034 relative to the left side of user 1's heart, albeit in smaller, lighter, and more water-resistant form than that of communication apparatus 700 and 900.

In a therapeutic setting, the above-described sensor arrays of each communication apparatus 700, 900, and 1000 may be operable with any other communication apparatus described herein to administer energy prescriptions. As shown in FIG. 1, for example, the physiological data output from the above-described sensors of communication apparatus 720, 920, and 1020 may be utilized in communication system 10 to adjust a therapeutic energy output from a plurality of haptic energy generators 773 and/or an element of any other data communication device described herein, such as haptic generator 240 of data communication device 220, haptic generators 340, 343 of data communication device 320, haptic generator 440 of data communication device 420, and thermoelectric generator 640, and/or LED 660 of data communication device 620. For example, each controller of apparatus 700, 900, and 100 may be operable with its respective sensor array 730, 930, or 1130 to determine cardiovascular measures associated with user 1, such as heart rate and blood pressure, listening to user 1's heart and lungs, predicting cardiovascular measures of user 1 based on the recorded data, and to modify the energy prescription accordingly.

### Graspable, Wearable, and/or Floor-Mounted Communication Apparatus

Exemplary aspects of this disclosure are now described with reference to communication apparatus 800. Aspects of communication apparatus 800 may be like those of other communication apparatus described herein but within the 800 series of numbers. Any elements described herein with reference to another communication apparatus may be similarly described with references to apparatus 800. Certain differences are now described with reference to apparatus 800 and its unique capabilities within communication systems 10, 20, 30, and/or 40.

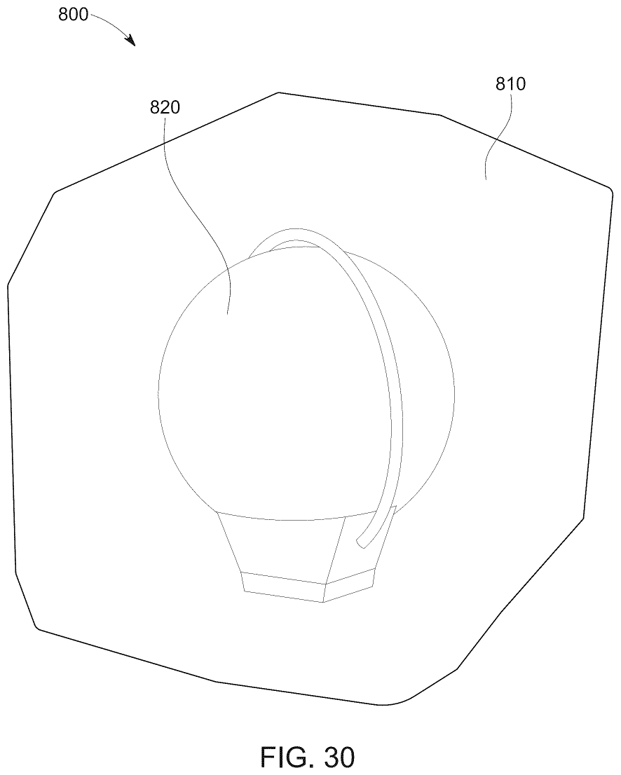

As shown in FIG. 30, communication apparatus 800 may comprise a housing 810 and a data communication device 820 in housing 810. Like housing 121 and its counterparts, housing 810 may comprise a biocompatible structure made of a material that surrounds and electrically insulates elements of data communication device 820, renders data communication device 820 waterproof to protect those elements against water damage from perspiration or elsewhere, and/or provides device 820 with a degree of impact resistance. Like housing 910, housing 810 may be operable to conduct and/or transfer one or more different haptic energies output by data communication device 820. Housing 810 also may comprise a semi-transparent and/or heat transferring material formed around data communication device 820. The material may comprise one or more base materials and different types of additives that are mixed with and/or suspended in the base material(s) to enhance a functional and/or visual characteristic of housing 810 when data communication device 820 is activated, making housing 810 a uniquely protective and communicative feature of communication apparatus 800.

Housing 810 is shown in FIG. 30 as having a resting surface operable to generally maintain a position of communication apparatus 800 when resting on the ground as best shown in FIG. 1. Housing 810 has a cube shape in FIG. 30 although it may assume any shape with an equivalent resting surface. The base material and the additives may be formed together to make housing 810 into a desired shape using any known forming, molding, and/or printing methods compatible with said materials. For example, the base material may comprise any type of semi-transparent crystalline material operable to receive thermal energy (e.g., heat) from data communication device 810 and distribute the heat to exterior surfaces of housing 810, including any known type of acrylic, glass, plastics, resins, silicones, and/or thermoplastics. As shown in FIG. 30, the base material may have a measure of radio transparency permitting wireless data communication signals and inductive charging signals to reliably pass to and/or from data communication device 820.

Aspects of housing 810 may be like “self-supporting body 633” of International PCT Patent App. No. US2021/030528, filed May 3, 2021, the entirety of which is hereby incorporated by reference into this application. For example, the polymeric material may comprise any one or more of the following: (i) a percentage of light refracting materials, such as glass or crystals; (ii) a percentage of light blocking materials, such as pigments; (iii) a percentage of glowing materials, such as a mixture of bioluminescent materials; (iv) a percentage of thermally reactive materials, such as stone powders or fragments; (v) a percentage of precious or semi-precious gemstones, fragments, or powders; (vi) a percentage any other crystalline structures, such as amethyst, selenium, quartz; a percentage of any other organic materials, such as bone, leaves, wood, or any other carbon remainders; (vii) a percentage of paper materials, such as a canvas or portions thereof, paper or portions thereof; and/or (viii) a percentage of any light modifying materials, such as films, glitters, and the like. As shown in FIG. 30, the additives also may have a measure of radio transparency that permit wireless data communication signals and inductive charging signals to reliably pass to and/or from data communication device 820. For example, if the additives have any metallic materials or function similarly, then they may be distributed and/or localized to ensure that one or more exterior surfaces of housing 810 is radio transparent.

Housing 810 may comprise a layer of the semi-transparent and/or heat transferring material that fully or partially surrounds data communication device 820. As shown in FIG. 30, the layer may fully surround data communication device 820 so that it is fully enclosed within housing 810. Housing 810 may act as a protective barrier. For example, a thickness of said layer of material may be optimized to provide data communication device 820 with an appreciable degree of drop protection, corrosion resistance, and/or moisture resistance. As a further example, housing 810 may render data communication device 820 waterproof, allowing communication apparatus 800 to be gripped by sweaty hands, placed in orifices, and/or even used underwater with a minimal risk of damaging device 820.

Communication apparatus 800 can be provided with many different housing types and styles. As shown in FIG. 30, housing 810 may be a mass-customized aspect of communication apparatus 800 that is manufacturable according to the beliefs, tastes, and material desires of its purchasers. For example, it is contemplated that most purchasers will prefer a more modest version of communication apparatus 800, such as one with a hard plastic shell, whereas others will prefer more elaborate and/or expensive options, such as by incorporating precious gems. In each of the depicted examples, the basic shape of housing 810 and its base material may be similar whereas the additives may be different. Some differences may be functional in nature, such as when certain heat conductive materials are selected for the purpose of increasing a heated duration of data experience apparatus 800 in use, or when certain crystalline materials are selected for the purpose or reacting with select frequencies of light and/or vibration (e.g., in ways real and perceived). Other differences may be aesthetic in nature, such as when the additives include a certain pigment and/or arrangement of pigments selected for the purpose of creating a desired visual effect, such as by incorporating a pattern or visual design into housing 810.

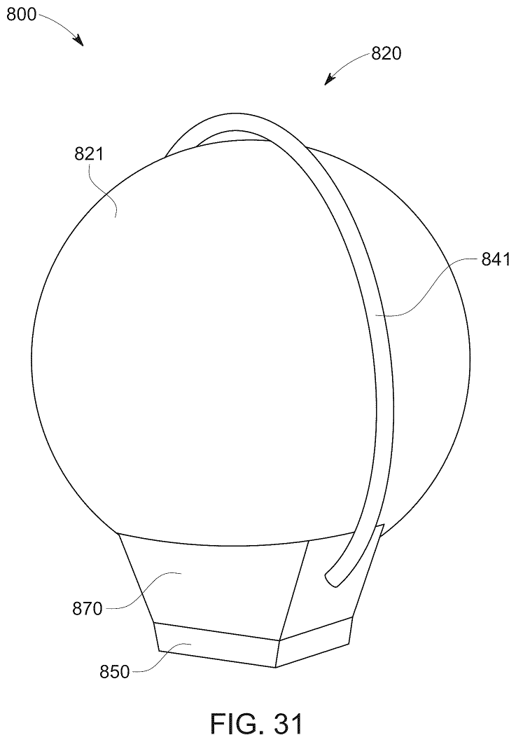

As shown in FIGS. 30, 31, 32, 33, 34, and/or 35, data communication device 820 may comprise a PCB 822, an exterior shell or lens 821, an interior frame 823, a power source 824, a controller 825, a data transceiver 826, a sensor 830, a haptic generator 840, an inductive charging coil 850, an LED 860, a heat transfer element 870, and/or an energy transfer element 880.

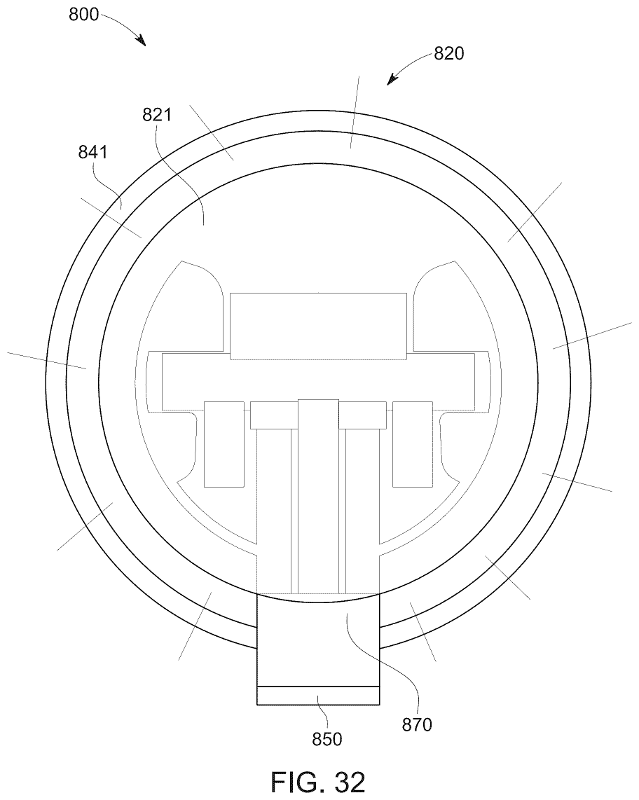

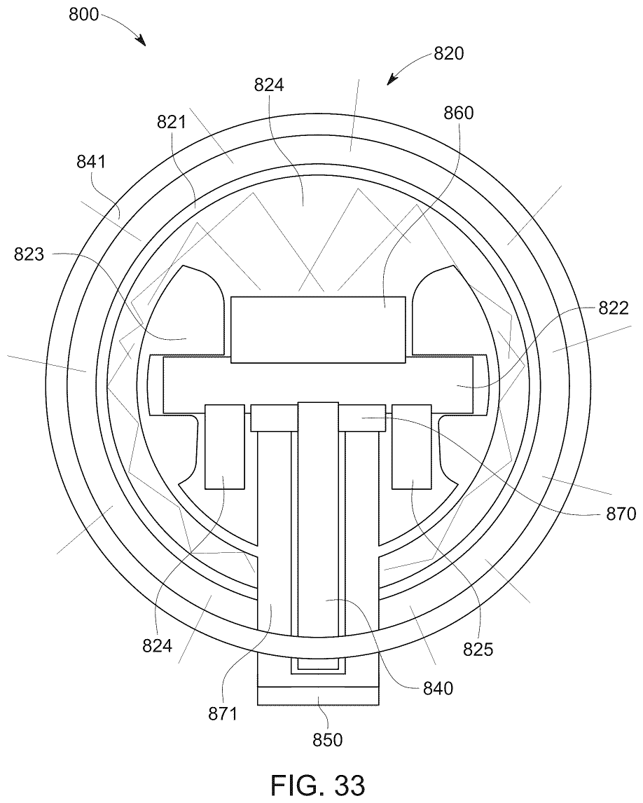

Exterior shell 821 may comprise a spherical or semi-spherical shape that defines an interior cavity and fully or partially surrounds interior frame 823. As shown in FIG. 32, exterior shell 821 may comprise a semi-spherical shape that defines an interior cavity that at least partially surrounds frame 823. Exterior shell 821 may comprise an exterior surface that is bondable with the semi-transparent and/or heat transferring material of housing 810 to hermetically seal the electronic components of data communication device 820 inside of shell 812. For example, the exterior surface of shell 812 may be rough or intentionally roughed to diffuse light more evenly into housing 810 and provide additional surface areas for bonding with the semi-transparent and/or heat transferring material of housing 810. As shown in FIGS. 32 and 33 with thin black lines, exterior shell 821 also may comprise an interior surface that is polished and/or chemically altered to distribute light more evenly into the interior cavity, such as by diffusing the light, scattering the light, and/or by permitting a first percentage of photons to pass therethrough and reflecting a second percentage of the photons back into interior cavity for subsequent distribution.

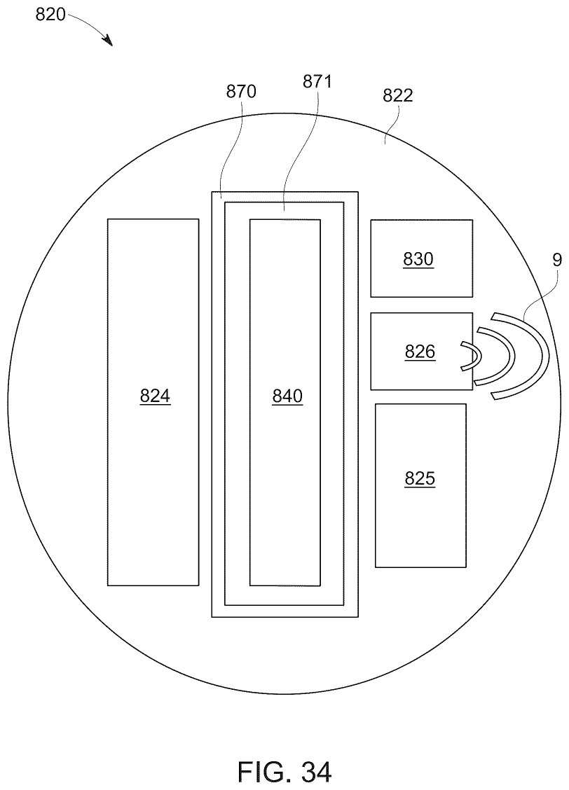

PCB 822 may be mounted to frame 823 and contained in shell 821. In keeping with above, PCB 822 may comprise a structural frame including conductive tracks, pads and other features etched from one or more sheet layers of copper laminated onto and/or between sheet layers of a non-conductive substrate. As shown in FIGS. 33, 34, and/or 35, power source 824, controller 825, data transceiver 826, sensor 830, haptic generator 840, LED 860, and/or frame 823 may be electrically and/or structurally connected to PCB 822. LED 860 may be engaged with a first side of PCB 822, as shown in FIGS. 32 and 34; and power source 824, controller 825, data transceiver 826, and haptic generator 840 may be engaged with a second, opposite side of PCB 822, as shown in FIGS. 33 and 34. Sensor 830 may be mounted to PCB 822 or located elsewhere on data communication device 820. Frame 823 may be engaged with both sides of PCB 822.

As shown in FIGS. 33 and/or 34, power source 824 may comprise an inductive charging coil 850 and a rechargeable battery. As shown in FIGS. 30-33, inductive charging coil 850 may be positioned at one end of housing 810 for transmission of power between coil 850 and a charging pad when housing 810 is placed on the pad. The rechargeable battery may comprise a lithium-ion battery mounted on PCB 822. All or at least a portion of housing 810 may be configured to permit the transmission of power to coil 850.

Controller 825 and data transceiver 826 may be operable as described above, meaning with any combination of technologies for receiving, processing, and/or sending data between data experience device 800 and another device. As shown in FIG. 34, controller 825 may be operable with data transceiver 826 to output data from sensor 830 to computing device 11 and/or receive control signals from computing device 11 over a wireless network 9 such as Bluetooth or Wi-Fi, either of which may pass through housing 810.

Sensor 830 may comprise one or more sensing elements that are operable with controller 825 to capture sensory data and output the sensory data to external control device 6 through housing 810. For example, sensor 830 may comprise any one or more of (i) an inertial measurement unit or IMU that is contained in interior cavity 824 and operable to output sensory data associated with a specific force, angular rate, and/or orientation housing 810; (ii) a microphone and/or camera that is positioned on or adjacent exterior shell 821 and operable to output sensory data including audio and/or visual data associated with activities outside of housing 810; and/or (iii) another type of optical sensor that is mounted on frame 823 and operable to output sensory data regarding light levels in and/or outside of housing 810. The one or more sensing elements of sensor 830 may comprise any combination of these technologies. The sensory data output from sensor 830 may thus be utilized to determine whether housing 810 has been moved, exposed to certain environmental stimulus, and/or is functioning properly, providing an important means of data feedback associated with the usage of data experience apparatus 800.

Haptic generator 840 may comprise any type of vibration-generating device operable to cause housing 810 to vibrate at a desired frequency. As shown in FIGS. 33 and 34, haptic generator 840 may comprise a coin-disc vibrator like those sold by Precision Microdrives or any other type of electrically powered mass-moving device operable with controller 825, exterior shell 821, and/or frame 823 to output a desired range of vibrational frequencies responsive to control signals from controller 825, including the examples described above. As shown in FIG. 34, haptic generator 840 also may comprise a surround 841 that reflects heat away from haptic generator 840 and/insulates generator 840 from heat exposure.

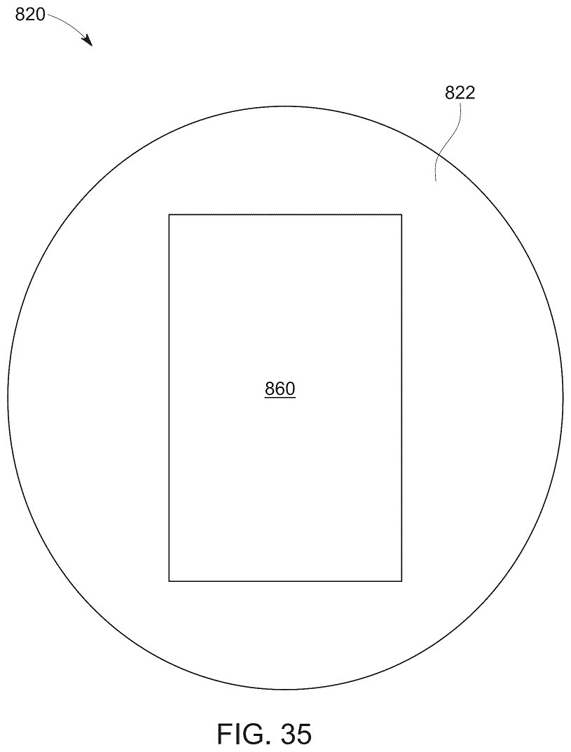

As shown in FIGS. 33 and 35, LED 860 may comprise a light emitting diode(s) that is mounted to one side of PCB 822 and operable with controller 825 to output frequencies of light into interior cavity 824 of exterior shell 821 for distribution into housing 810 through shell 821. LED 860 may comprise a multi-color LED, such as an RGB LED operable to output millions of different colors at varying intensities. As shown in FIG. 33, LED 860 may be mounted to PCB 822 with heat transfer clement 870, which may comprise a block of heat conductive material (e.g., aluminum) that extends from a back, heat transfer surface or LED 860 and through PCB 822 to establish a thermally conductive attachment to frame 823. Heat transfer element 870 may engage LED 860 with PCB 822 to limit that amount of heat retained by PCB 822. For example, heat transfer clement 870 may be provide a direct path for transferring heat from LED 860, through PCB 822, and into frame 823, minimizing the amount of heat flowing through PCB 822. As shown in FIG. 33, edges of PCB 822 may be contained within recesses of frame 823 to provide a complementary, indirect path for transferring heat from LED 860, through PCB 822, and into frame 823 via interior surfaces of the recesses, minimizing the amount of heat retained by PCB 822. As shown in FIG. 35, LED 860 may be mounted on one side of PCB 822 to ensure that is spaced apart from any electronic components mounted to other side PCB 822, creating an insulative air gap therebetween.

Frame 823 may comprise a complex 3D structure operable to position the elements of data communication device 820 relative to one another. As shown in FIG. 33, an upper portion of frame 823 may comprise legs defining recesses for containing edges PCB 822 therebetween, centering it within shell 812; and a central portion of frame 823 may extend between the conductive attachment to heat transfer clement 870 and an inductive charging coil 850. The conductive attachment may define an interior cavity and haptic generator 840 may electrically and structurally connected to PCB 822 at a location inside the thermally conductive attachment. Surround 841 may comprise a thermally insulative epoxy located between interior surfaces of frame 823 and exterior surfaces of haptic generator 840. As shown in FIG. 33, energy transfer clement 880 may comprise a ring that surrounds shell 821; and a portion of the ring may be thermally coupled to frame 823 and vibrationally coupled with haptic generator 840. For example, said portion of the ring may extend through holes in bottom portions of frame 823 and haptic generator 840 and soldered or epoxied therein to create a rigid connection operable to transfer heat input to frame 823 via LED 860 and/or vibrational energies from haptic generator 840 into housing 810, where it can be experienced by user 1 and/or dissipated into ambient air to prevent device 820 from overheating.

As shown in FIGS. 1 and 30-35, the shape, size, data-gathering, and energy-outputting capabilities of communication apparatus 800 may make it uniquely suited for gathering particular types of sensory data from user 1's environment and administering energy prescriptions to user 1 by light, chemicals, and/or aromatic therapeutic elements into the environment. For example, when placed adjacent user 1, aspects of communication apparatus 800 may be operable to (i) measure and output sensory data associated with user I by tracking changes in their ambient environment, such air, light, sound etc.; and (ii) prescriptively affect user 1's physiological data by causing haptic generator 840 and/or LED 860 to output vibratory and/or optical energies toward a location external to user 1's body responsive to the sensory data. When grasped in hand 5, the communication apparatus may be similarly operable to one or more different energy types toward skin of user 1, such as optical, thermal, and/or vibratory energies. Advantageously, because housing 811, heat transfer element 870, and energy transfer element 880 are thermally conductive elements, communication apparatus 800 may be operable to output heat without using a thermoelectric generator.

Because the electronic components of data communication device 820 are hermetically sealed in housing 811, and because the entirety of housing 811 may heat up, light up, and/or vibrate, communication apparatus 800 also may be operable to diffuse aromatic and/or chemical therapies into user 1's environment. For example, communication apparatus 800 may be dropped into a volume of aromatic wax and/or medicinal chemicals (e.g., a mixture of camphor, eucalyptus oil and menthol) so that housing 811 is fully or partially submerged. In this example, controller 825 and/or another processor in data communication therewith may activate haptic generator 840 and/or LED 860 for the purpose of creating a unique visual display, heating the volume to release its aromatic and/or chemical compounds, and vibrating the volume to further release its aromatic and/or chemical compounds, or to signal that more should be added.

As shown in FIGS. 1 and 30-35, for example, certain advantages and performance benefits also may be realized with by adding one or more communication apparatus 800 to communication system 10. For example, each communication apparatus 800 may be operable to affect the environment of user 1 by outputting light, aromatics, and/or chemicals in their vicinity. As a further example, one communication apparatus 800 may be held in each hand 5 of user 1 and operable with the unified control signal to output optical, thermal, and/or vibratory energies toward user 1's skin and/or eyes.

### Wearable Communication Apparatus

Aspects of exemplary communication apparatus 1200 are now described with reference to FIG. 1. As shown therein, communication apparatus 1200 may comprise a plurality of body mounted, wearable, or implantable haptic technologies like those described in U.S. Pat. No. 10,959,674 issued Mar. 30, 2021, and International Patent Application No. PCT/US21/57634, filed Nov. 2, 2021, the entireties of which are hereby incorporated by reference into this application.

### Fabric-Integrated Communication Apparatus

Aspects of exemplary communication apparatus 1300 are now described with reference to FIG. 1. As shown therein, communication apparatus 1300 may comprise a plurality of a plurality of data communication devices, an electrical network operable to transmit data and power between the plurality of data communication devices; and a fabric-mesh structure comprising an insulating material shaped to house the plurality of data communication devices and the electrical network, maintain a plurality of air gaps between the plurality of data communication devices, and define a skin contacting surface that is directly engageable with the skin, similar to those described in International Patent Application No. PCT/US21/57634, filed Nov. 2, 2021, the entirety of which is hereby incorporated by reference into this application.

As described above, communication systems 10, 20, 30, and 40 may comprise a plurality of energy outputting technologies operable to repeatably and reliably deliver an energy prescription(s) to a user by causing a plurality of communication apparatus located on or adjacent user 1's body to simultaneously output different types of energy toward different parts of user 1's body. The output may occur in a coordinated matter during a treatment as part of a synchronized communication of the different types of energy to different sets of nerves and tissues in order to affect user 1's brain and/or mind by inducing different mental states, overwhelming the senses, or otherwise changing user 1's brain activity with the responsive to a unified control signal to the plurality of communication apparatus.

Different environmental and/or wearable communication apparatus may be utilized to output the different types of energy with communication systems 10, 20, 30, and 40, such that the plurality of communication apparatus may comprise any combination of exemplary communication apparatus 100, 200, 300, 400, 500, 600, 700, 800, 900, 1000, 1100, 1200, and/or 1300 described herein, each of which may be utilized with one of communication systems 10, 20, 30, or 40 to output one or more different types of energy toward user 1. In keeping with this disclosure, the unified control signal may thus be operable to coordinate the plurality of communication apparatus together so that they function as one medical device with disparate components, offering a combined benefit exceeding that of any apparatus by itself.

Numerous sensing technologies are described with reference to communication apparatus 100, 200, 300, 400, 500, 600, 700, 800, 900, 1000, 1100, 1200, and/or 1300 as being operable to capture physiological data associated with user 1 during the treatment period. For example, it is contemplated that the physiological data may be captured from any combination of sensor 130, sensor 330, sensor 430, sensor 630, sensor array 730, sensor array 930, and/or sensor array 1030. Numerous computing technologies are described with reference to communication apparatus 100, 200, 300, 400, 500, 600, 700, 800, 900, 1000, 1100, 1200, and/or 1300 and/or external device 11 as being operable to analyze the physiological data for the purpose of confirming that the energy prescription was able to produce a desired or intended result during the treatment period. For example, if communication system 10, 20, 30, or 40 is being utilized to administer an energy prescription intended to have a calming effect on user 1, then these computing technologies, with or without external device 11, may be operable to verify that the energy prescription is having or had the desired effect on user 1 by confirming that they did not experience an accelerated heart and/or breathing while the different types of energy described herein are being simultaneously output to their body.

Nonetheless, while described as part of communication system 10, 20, 30, and/or 40 with reference to certain combined benefits, it is contemplated each of exemplary communication apparatus 100, 200, 300, 400, 500, 600, 700, 800, 900, 1000, 1100, 1200, and 1300 may be operable as a stand-alone device. For example, to realize their intended benefits, some energy prescriptions may require a more wholistic experience involving a combination of outputs from apparatus 100, 200, 300, 400, 500, 600, 700, 800, 900, 1000, 1100, 1200, and/or 1300 whereas other energy prescriptions may be successfully administered with sustained outputs from just one of these apparatus over a period of time.

While principles of the present disclosure are disclosed herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects disclosed herein. Accordingly, the present disclosure is not to be considered as limited by the foregoing description.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A data communication apparatus comprising:
electronic components operable to gather and output different types of physiological measurements associated with a user's chest, the electronic components comprising a multimodal sensor array, a transceiver, and a power source; and
a housing that contains the electronic components and comprises an attachment element operable to maintain an alignment of the multimodal sensor array over the user's chest,
wherein, when the housing is maintained over the user's chest, the multimodal sensor array is operable with the power source to measure the different types of physiological measurements, and the transceiver is operable with the power source and the sensor array to output the different types of physiological measurements to an external device.

2. The apparatus of claim 1, wherein the multimodal sensor array comprises left sensors located on a left side of the user's chest and right sensors located on a right side of the user's chest.

3. The apparatus of claim 1, wherein the multimodal sensor array comprises two or more of:
a first sensor operable to measure a first type of physiological measurements associated with the user's heart;
a second sensor operable to measure a second type of physiological measurements associated with the user's lungs;
a third sensor operable to measure a third type of physiological measurements associated with the user's head; and
a fourth sensor operable to measure a fourth type of physiological measurements associated with the user's body.

4. The apparatus of claim 3, wherein the first sensor comprises electrodes positioned to measure the first type of physiological measurements.

5. The apparatus of claim 4, wherein:
the electrodes comprise left electrodes located on a left side of the user's chest and right electrodes located on a right side of the user's chest; and
the first type of physiological measurements comprise a left measurement for the left side of the user's chest and a right measurement for the right side of the user's chest.

6. The apparatus of claim 3, wherein the second sensor comprises a transducer positioned to measure the second type of physiological measurements.

7. The apparatus of claim 6, wherein:
the transducer comprises a left microphone located on a left side of the user's chest and a right microphone located on a right side of the user's chest; and
the second type of physiological measurements comprise a left measurement for the left side of the user's chest and a right measurement for the right side of the user's chest.

8. The apparatus of claim 3, wherein the third sensor comprises an input device positioned to measure the third type of physiological measurements.

9. The apparatus of claim 8, wherein the input device comprises a camera positioned to measure the third type of physiological measurements.

10. The apparatus of claim 9, wherein:
the camera comprises an upward-facing camera; and
the third type of physiological measurements comprise data associated with the user's face.

11. The apparatus of claim 9, wherein:
the camera comprises a forward-facing camera; and
the third type of physiological measurements comprise data associated with the user's environment.

12. The apparatus of claim 3, wherein:
the fourth sensor comprises an inertial measurement unit, a movement sensor, or a kinetic energy sensor; and
the fourth type of physiological measurements comprises data associated with a movement of the user's body.

13. The apparatus of claim 1, wherein the housing comprises a radiotranslucent material and the transceiver is operable to output the different types of physiological measurements to the external device through the radiotranslucent material.

14. The apparatus of claim 1, wherein a portion of the housing is translucent.

15. The apparatus of claim 1, wherein the electronic components are hermetically sealed in the housing to prevent exposure to water when removably attached to the skin.

16. The apparatus of claim 1, wherein the attachment element comprise a skin-facing surface of the housing with a biocompatible adhesive layer operable with the skin to maintain the alignment of the multimodal sensor array over the user's chest by acting on the skin.

17. The apparatus of claim 16, wherein the skin-facing surface is conformable against the user's chest to form an adhesive bond between the biocompatible adhesive layer and the skin.

18. The apparatus of claim 17, comprising flexible conductors that are embedded in the skin-facing surface and operable to establish a data connection between the sensor array and the transceiver.

19. The apparatus of claim 18, wherein:
the multimodal sensor array comprises left sensors located on a left side of the user's chest and right sensors located on a right side of the user's chest; and
the transceiver and the power source are located between the left sensors and the rights sensors; and
the flexible conductors comprise left conductors connecting the left sensors to the transceiver and the power source and right conductors connecting the right sensors to the transceiver and the power source.

20. The apparatus of claim 1, wherein the attachment element comprises an elastic portion that is operable with the user's torso to maintain the alignment of the multimodal sensor array.

21. The apparatus of claim 1, wherein the attachment element comprises a lanyard that is operable with the user's neck to maintain the alignment of the multimodal sensor array.

22. The apparatus of claim 1, when the housing is maintained over the user's chest, the multimodal sensor array, the transceiver, and the power source are operable with the external device over a wireless network to determine a cardiovascular measure associated with the user's chest.
